SEQUENCE LISTING (16 sequences)

SEQ ID NO: 1 - DNA, 5149 bp, rattus sp.
tcccacttcagagcggccgaggccgagggcctggagcaggccgcgctgccggccgaggagagccgtgtgcagcccatgaccgcctccaactggacgctggtgatggagggcgagtggatgctgaaattttatgctccatggtgtccatcctgccagcagactgattctgaatgggagacttttgcaaagaatggagaaacacttcagatcagtgtgggaaaggtggatgccattcaagaaccaggtttgagtggccgcttctttgtcaccactctcccagcattttttcatgcaaaagatgggatattccgccgttaccgtggtccaggaatctatgaagatttacagaattacatattagagaagaaatggcaatcagttgagcctctcactggctggaagtccccagcctctctaacgatgtctggaatggctggtctttttagtatctctggcaagatttggcatcttcacaactatttcacagtgactcttggaattcctgcatggtgttcttatgtttttttcgtcattgccactttggtctttggcctgttcatgggtttgatcctggtggtgatctcagaatgcttctatgtgcctcttccaagagcttcctctgaacgctctgagcaggagcagaggtctgaggaggctcaaggagctgagcagctccaggatgcagaggaagagaaggatgactcaaatgaagaagaaaacaaagacagtcttgtggatgatgaggaagagaaagaggaccttggagatgacgatgaagtagaagaagatgaggaagaagacaacttggcaggcatcatggatgaggagaaaagtgacagcaatgagcaaggagttgtgaaggagggcagtgtgtccccgaaggatgaagaagctcgttcagctgacacacaggacgtggtggaagactccttgaggcagcgcaagagtcagcatgccaagggaccataggttacattatgctacttctaaaaatacaggccaaaaaaaaataagtttgtttccctctggtgtgcagtttaagccaaatccttagttgtttgttttgttttgtttctctggatgagcagtgttcacttttaaaagatgatctctggccctgtggttacgtggtagtaagctcagtgttgtaagagtcaggtgcagcacttagccaccatggtgtcaggatctttgtggggacttgtgcagaacaagtcgatgtgcttgggaactaaggagtaaatcagaagacttctctgcagttctcatgagcacctttagaaatgaggctgctgcttagtgaagtgagaacaaagccaccgcactctggagcaccttgaagtagaggaagtgtttgccgtcaccgttaagcatgtcctttagtcatgtcagcaacataggtgccacagctccacaacattatcttggggttagaaattgtggagacctggatatagatacaacttcaaaatgaagtcatgtaagctcttggggtctctgttgtcttttgtttcttacctttaatctctatgacttttcctctttgacttgcagactccatttcacagctgctctgtagtgagaagagcaggcagcagtgatctgcatgcgttacccttaattgaagtaaagcttgaggtaatcagtaatcacagctgatgatgccaagaccgccaagtgtctcaaagatatgctgtactctgaaaaagcaaaagagagtagtggattcataggacaggtataaacaacatcttgcttgggtccctatagttacaggatgacttctttaagtttcttcagccacattctttgtttttctgaaccgaaagtgctctatatcttgtcactacaaggtagttttctaaagctgacttcatgtctgtgatagtttccatctccaggaatctctctggaatcactttaataaccactgaaaaggcatgtcggctttgcttttcttgatgtgcttttggtgaaaaaattgatgaaagtcaatatcaagaggtgaaagatttgatttgtttctatctcccttgatcctctagagaattgtatgattataaatctctccctgctcaatctaatatcagtgtcaaggaacccaactttcttaaaaaaatatttccatctcctctcttaccctctcatgtctgcaaataaatgcataaaatagattttagctgtatgaaagatgactaaagtgaatcttattacccttgggttgagtcagagtaatgagtttaggtttgtgaaattcaaaatgtttcgacattttaaaaagggatatgtagtgtaattctctgtctgcttagataaccctttgctgccaacaggagagtcattggtagtggaggttcagaattcgccctcctatactgcacaagcctgctacaaactttgcacaaaggaagattttattgtgacaatccattctttatcaatgtttttatttcatctttgtaacttagttaactatcagttcaaggctaaggctatcacagaactggagacctacttcccttccatgtttctgcccttctgatacatccttactcaaaagtgccatggttctgtggtacttcctgctatatcttcctactagagctaagctgcaccttctcttttgcagtgcattttgtttccttgtaatcagatgcatggagtgtgtaatagtaaatctatgttcagggtatggaaaagggaaggcttgtgtctctgttctgtgacgtaccctcctccaaattgacatactgggttctagtgcgataattgagcagaggaaaacttgaagacttacagtcagccttagctttgataaatgttttaaaaccacattccagttggaccacaaatactatctaataatataatgagataatatattttctttcttaataatacttaaaagatattggaggctgggagaaagggccacgaagaagaggcctaaaattatatttatttgtaggaacaaatttttcctagcttttctctttttaatatcctggtaaaaattgggtttctagtaaatagtccaggagtatttacttaacttaatttcttttgatttaaagcctttaggttaattttcaatactttgttaatataaaattaaagggaaagaccaacttacgattagatcaacatttaatatcactttatttttgtgtatataataaatttaatccagaagttaagtgcatgaaaacatgattttataaaacaaaactaaccatgcttcccctccattgccatacctgctatttaattgtagattgctattttctgtatgttaaaattgggtgaatgagatagcaattgtggaatgaggatatatagtgctttatggacaggtagttagagaaaggtgccttgttcttcattggaatatctgaaaattccacctctttttaatttttcttagcacttcaccccttaaccatgctgaaccatagcagcttgcttagaatctcatggaagcaatgcacaggcaaattcacgtttttgttgttttgattgttttgattttgaaagctaatcgtggaacatggaagagatggctcattcagtgcacaggccatttcgctgggtgctgtggcagtcactgcagtctcatcctatacataggctctgtacctattactatagtaactagtgcagtggaaggcaaacacgcttgacaagatcgtctctgggtagctgaaaagctacactctcacattttagggccaccccatgactgataattaagatactgctgtgtagtttgagtatgtgtaaacttgaataagagcaccttgtaacatcttttgacgaggagattaaaatgctagttaacagaatatctaccatgttgctttttgcatgatgggaaacaatggagttcaaagggaagttttaaaggaatgaaaagggtgaaagatatttcagcattaaaatcctaagcatgaagtgaaaggagagattggtacttctgctggtgtatacaggttttctgtgttcttctcttagtgttccgctgttattcctgccagtattgtacatgcgtgattgacttgcctgtcaaatctatcaagtattggaaagtatttgtgtaaatctttgtgccaacaagaattctcccagtgttgtgtatctttacctcataccattcattgatatggttcttgtgagattgtagctcgtctgacagaagccaaggtggaatccgatgaaacctgccgaggcaggaggttcaactgagaaacctaagacagcttcagcctagcatgtggacctcagcggcatagtctctaaagttttgtgtaaactgtgatgttcacctgtggctttgacccatttctgatgtggtttaattttatttctttctggacagctttgtagaaatggaatttttatactaaagacactgttcacttgtaagtcaatacattataaaatggtgtatttattttttgtaatagtgtggattgtttagcccagtgtatttattttcctttacctgtgtattctgtgctctcaaatagatgtgtagcaattgaaatgagaactcagtgcatcatttcaagcactacattgcctgcattgcaaattctctgagcacaggattgtacagaaccctgagcctgtgtactgccatttgattgtcagttccatttgtgtcaaaacctaaaggtggtgatgaccaagtgaaacagagtgctgtactgctttttcatatgaatattttcacataaaagcatttgtgaaagcagttgaaaatattactttgtttaaaaaaatctttcttctgtgttyaaaaaaararwattcattctctacctttcatacctaaacttggtgaaattcatgccatgtgcccaaggttaatgctgaagatgtaaggccttctgaacacaccacagctcattagctcactgtactctctgtacgagctcgttccacttgccatggccaggttggtgcagttactaagtaactctgctactcctccaagattgctggacttccgcattcttctaggacttctagaagaccatctcaacttaaccgttcccaccactgtagcaataaaaaaaggaaagggcgggggctcctttgaacacacttaaaaataaaaaaaaaaaaaaaaaaaaaaaaaaaaaaaaaa

SEQ ID NO: 2 - protein, 322 aa, rattus sp.
SHFRAAEAEGLEQAALPAEESRVQPMTASNWTLVMEGEWMLKFYAPWCPSCQQTDSEWETFAKNGETLQISVGKVDAIQEPGLSGRFFVTTLPAFFHAKDGIFRRYRGPGIYEDLQNYILEKKWQSVEPLTGWKSPASLTMSGMAGLFSISGKIWHLHNYFTVTLGIPAWCSYVFFVIATLVFGLFMGLILVVISECFYVPLPRASSERSEQEQRSEEAQGAEQLQDAEEEKDDSNEEENKDSLVDDEEEKEDLGDDDEVEEDEEEDNLAGIMDEEKSDSNEQGVVKEGSVSPKDEEARSADTQDVVEDSLRQRKSQHAKGP

SEQ ID NO: 3 - DNA, 3308 bp, rattus sp.
tttcgcctgtttttggttttgtgacttagaggagagaacttttctaagcaggccgggcacgactgaaggctactctgcccccacgtggacgctcccctggggaaaagaagtctgaaagtaaacttagcctcgccggcctgaaggcctcaactttggctggactgtaagtttcggagtcatgaccttctcgctgggcaggtgggaatctttatctatcttgggctggacctgaaacctcagtcagcacccaaatctcccccaccccattcacactttgtaccaggggtaggccccggacactgatctacaacatggataaatatgatgatctgggccttgaggccagtaagttcattgaggacctgaacatgtacgaggcctccaaggatgggctcttccgggtggacaagggtgctagcaacaaccccgaatttgaggaaactcgaagggtgttcgcgaccaagatggccaaaatccacctccagcagcagcaacagcagcagctcctacaggaggaggccctacctagggcaggcagaagccccatcaacggtgggaaccgtcagggtgtgagcagcaagctggctgcagatggggccgctaagcctcctcttgctgtgccaacagtagcacctggactagctaccaccactatggcggtgcagtcctcatacccacctcaggagcagaggaccaggccatctgcccacggtgcaaggcctggcagtcagaactgtggttccagggaggggcctgtgagttcccagagacctgctttacacggtctgggtccctgtgaagacccttcctgcctcactcacggagactattacgacaatttctctttggcaagcccacagtggggtgataaaccagaagagtcccctagcatgagtttgagtgtagggagtggatggcctggttgcccaggaaatgattccctgtcgcacagatcctgtggggacagtcatccttaccacccacagctctccatgtgctctggcaggtcttttgaaagtggtcaggacagtggcattggtggccatagcagtgagaagccaacaggcctttggtccactgcctcctctcagagagtgaacctgggcttttcttccacgggcttggagaatgggaccccagctcaacccaagggcacaaccgtttcagcaccaatggtccctagcagcactagccaaggcgcttgtctgagaagagattcgagtctgggatatgaggctccaggcagggtcttcaaacctcttgtggacactcagccttggctccaggatgggcccaagtcgtacctctctgtttctgctccactatcctcaacaaccagcaaggacaatgccaagacaggtatgaccgctggactggatcctaagcttggatgtgtggagtctggcactagtcccaagcccagccccaccagtaacgtccatccggtaatgtctgctccgtctgagttatcttgtaaagagagtcctcccagctggtccactgacagtagcctgggacctgtgctcccagagagccccaccccctccagggtgaggttgccctgccagaccctcacgccaggccccgagcttggaccctccactgcggaattgaagttggaagccctcacccagcgtctggagcgagagatggatgctcaccccaaagcagactacttcggagcctgtgtgaaatgcagcaaaggggtgtttggagctggccaggcctgtcaggccatgggggatctctaccacgatgcctgcttcacctgtgcagcctgcagcaggaagttaagaggaaaggccttctattttgtcaatggcaaagtattctgtgaggaagactttttgtactctggctttcagcagtctgcggacagatgttttctttgtggacacctgattatggacatgatcctacaggccctagggaagtcctatcaccctggctgtttccgctgcgtcatctgcaatgagtgtctagatggggttcctttcaccgtggactctgagaacaagatctactgtgtccgagattatcacaaggtgctggcccccaagtgtgcagcctgtggccttcccattcttccaccagagggctcagatgagaccatcagagttgtgtccatggaccgagactaccatgtagagtgctaccactgtgaggactgtggtctggagctcaatgatgaggatggtcaccgctgctatccactgggggaccacctgttctgtcactcctgccatgtcaagagactggagaaaggaccctcacctgcatccctccaccagcaccacttctagcaaagagacagtggggatgtggggccagcctgcccagggaggtgtctgtgtagcacttctgagcttccccacagctggggcacaggaagaggagggacaggaagttgagttcctgtgtgtgtgaagggtatcattcttttaaccacagcagtgtgcactccccatcctgtccgtgtgttttccaagtgcttttctctattgtcacactctcgccaaatcactcaggaagatgctccaacttgcacatgacacaagatggggtctgtgtaactggacacatcctactccctggaaacaggttttcagattgtgttgggcatattccacagtgtccctaaagagaaacaatggtggaggtgagcaggagaatgcctgcacccagataaccctcacacagcctgtgctgtgacttcacagttctgtccacttcagagctttagttcttctgaggatgcttcaaacccttaactgcattacagcaggaaagaagtactgcatatatttgctttgtaccttggagtttgcttggtgaaagagagccaagcttagacatctgggaggctgggtctccctgctgtgcctctgcagttcctgctgcacctaaggcagtggagctggggcagaggcctgccctgccctgccctgccctgccctgtcctatatagccagctcagagagaactggtgcaggcaccaggcttcttgtccatgcttggtgctgtgtttcattgctttcctccagtctgtgacattggagttgcttcctcaaggcgagtctgaggatctcatgcgctcagatccctgtcaaaggttgggaatcctagtccatatcctgggagggagctccttccagaaggaaagtgttgtgtgtgagatctcaaagctagcaccagagggagtgcctgagcttgtttcacaggcatctctctcggccacctctcagcacagacattcgtttggtttgatgaaaccaatataaggtacactgctttaaaaaaaaaaaaaa

SEQ ID NO: 4 - protein, 663 aa, rattus sp.
MDKYDDLGLEASKFIEDLNMYEASKDGLFRVDKGASNNPEFEETRRVFATKMAKIHLQQQQQQQLLQEEALPRAGRSPINGGNRQGVSSKLAADGAAKPPLAVPTVAPGLATTTMAVQSSYPPQEQRTRPSAHGARPGSQNCGSREGPVSSQRPALHGLGPCEDPSCLTHGDYYDNFSLASPQWGDKPEESPSMSLSVGSGWPGCPGNDSLSHRSCGDSHPYHPQLSMCSGRSFESGQDSGIGGHSSEKPTGLWSTASSQRVNLGFSSTGLENGTPAQPKGTTVSAPMVPSSTSQGACLRRDSSLGYEAPGRVFKPLVDTQPWLQDGPKSYLSVSAPLSSTTSKDNAKTGMTAGLDPKLGCVESGTSPKPSPTSNVHPVMSAPSELSCKESPPSWSTDSSLGPVLPESPTPSRVRLPCQTLTPGPELGPSTAELKLEALTQRLEREMDAHPKADYFGACVKCSKGVFGAGQACQAMGDLYHDACFTCAACSRKLRGKAFYFVNGKVFCEEDFLYSGFQQSADRCFLCGHLIMDMILQALGKSYHPGCFRCVICNECLDGVPFTVDSENKIYCVRDYHKVLAPKCAACGLPILPPEGSDETIRVVSMDRDYHVECYHCEDCGLELNDEDGHRCYPLGDHLFCHSCHVKRLEKGPSPASLHQHHF

SEQ ID NO: 5 - DNA, 6573 bp, rattus sp.
gctaaatgaagtgtgcgcggaaccaagctcaaacacggaggcagaggcactgcggaaaagagcgatagaaggactgggtaggtaggctgcacgctgtcccccaggattatgaacctgtgaccgacactaagctgctccggctcacgagaatccacccacctaaaccaagacgggcaaaacagcagcagtcactcatctacagcgatcacgtccgggttcggccaccgcctgggcctctcgcttccaatagctgtcctcgcgcactcaagctgctcccgccaactcttcccaaacaagccgaagcggcgccggctgctaggcgacaccactccccgcccagccagccaaggccggaactaggccccggataccagggaagactacatttcacgactgggggaaaagctttacggctatgtttccccctccaagaccttttttcactgtggatgagggaaagccgtgagaagaggactctacgtcccagagtgcagttcggcgctgcggctgtgcatactgggattagaagttttgggaagagcagcgcgttcccggcgtgcaatgcgagaggctgcggccgccctggaagagctggggaaggatcgggaggcgggaggagcggctgctaaggtgcggccgcccaggagccggagcgtggcggactatgacagtgttttctgcagagtccttctccggtccaccgtctccgaagctttcctgaaccatctcttggcggagccgccctcagtttgcctggttaatgatggaagggaagatggctgatattaacttcaaagaagtgactttgatagtcggcgtggttgctgcctgctactggaacagtctgttctgtgggtttgtttttgatgatgtttcagcaatactggataccaaagacctgcacccatctacacctttaaaaactttatttcagaatgacttctggggaactcctatgtctgaggagagaagccacaagtcatacaggcccttaacagtgttgacatttcgcttaaattacctcctcagtgaactaaagcccatgtcttaccacctcctgaatacggtgttccatgctgtggtcagtgtgatctttctcaaggtctgcagactttttctggataagaggggcagtgtgattgcggctctgctctttgcagtgcacccaatccatacggaagcagtcacaggtgttgtaggaagagctgaactcttatcctctgtctttttcctagctgcattcctgtcgtatactaaatccaaaggaccagacaattccatagtgtggactccgattgcattgacagtgctcttagtggctgttgcaacattgtgtaaagaacaagggataacagttgtcggaatctgttgtgtatatgaagtatttgtggcccaagggtacactttgccattgttatgtgccgttgctggacagtttctccgcgggaagggtagcattccattttctatgctacagacattaataaaactcattgtcttgatgttcagtactttactacttgtcgtgatcagagtccaagttattcagtcacaacttccagtgtttacgaggtttgataatccagccgctgtaagcccaacccctacaaggcagctgacttttaactacctccttcctgtgaatgcttggcttctattgaatccttcagaactctgctgtgattggaccatgggaacaataccactgatagaatcatttctagatgttcgaaatcttgccacttttgctttcttttgctttctgggtgctttgggaatattcagtctcagataccctggtgactcctccaagactgtcctaatggcgctttctttaatggcgttaccatttattcccgcatcaaacctcttctttcctgttggatttgttgttgcagagcgagtactatatgttcctagcatggggttctgtattttagtagcccatggatggcaaaaaatttcaaacaaaagtgtcctgaaaaagctctcctgggtttgtctgtccatggtgatattaacccatgccttgaaaacactccatagaaattgggactgggagtcagaatatacattgtttatgtcagccttaaaggtgaataaaaacaatgccaaattatggaataatgtgggtcatgctctggaaaatgagaagaactttgagaaagctttgaaatactttttgcaggctacccatgttcagccagatgacattggtgcccacatgaatgtaggaagaacgtataaaaacttaaacagaactaaagaagctgaagaatcttacatgttggctaaatcactaatgcctcagattattcctggtaaaaaatatgcagccagaattgcccctaatcacctaaatgtttatatcaatttggccaacctcattcgagcaaatgagtcccgcctggaggaagckgaccagctgtaccgacaggcaatcagcatgaggccagacttcaagcaggcttacattagcaggggagaattgcttttaaaaatgaataagcctctcaaagcaaaagaagcatatcttaaagcactggagctggacagaaataatgcagacctgtggtacaacttggcaattgtttatattgaacttaaagaaccagatgaagctctgaagaactttaatcgggctttggaactgaactccagacataagctagcactgtttaattctgctattttaatgcaggaatcaggtgaagttaaactcagaccagaagctagaaaacggcttctaaattacataaatgaggagccgcaagatgctaatggctatttcaatttggggatgcttgccatggatgacaaaaaggactctgaagctgagacttggatgaagagggctatcaagttacagcctgacttcagaagtgctttgttcaacctggcactcctgtactcacagactgctaaggagttaaaggcattgccaatcttagaagagctgctcagatactatcctgatcataccaagggcctcattttaaaaggagatattctgatgaaccagaagaaggatatagctggagctaagaaatgttttgaaaagatcctggaaatggatccaagcaatgtgcaaggaaagcacaatctctgtgtcgtgtactttgaagagaaggacttgctgaaagcggagagatgcctagttgaaaccttggcattagcacctcatgaggaatatattcaacgccatttgagtatagtcagggataaaatcgcctcttctggtattgtggatcagccactgtccccagttgataagacttcaggtacagaagaaaaagatgaagtcccttccgaggatgtaaaagaaatcatcagtgagtccaggccagcacaaatcataaaaacaaatgataacagaaagtctcagtctaacaagcagtcaacaggaaatgcagaccaagatgccccccataaaacaacaaaagacatcaaagaaattgagaagaaaagagttgctgctttgaaaaggctagaagagattgaacggattttaaatggagaataacactaattctgtatcatgtaacaatgtagccaagaccttcagtctctatcatgtgtttgcttgtactggaaactgaaaaatgttgagactatatactggtttttcaaaaactgcataagattctctatataggatcaaaatgagatttttattgaagacctatctgtcccaggatatatgttttataagagaggacacaaattttaatttggaagaatattggagaaatcacaaattccaagtcacaacttgaaacrtttattatagaaagagaaagatgtgggtttattatagaaagaaaagggtggggaagtactgatatgtctttctactaatctgaattgaaataatatggggcatcaaagatgaacccttggagataaatccagaatgtactggggtttgtgaattcccatttattaactgtttttcatttttcattacctctttggattcaggaatggactgatactcattgaagacattcagtaactcttctcataaaccaagggacattatactgcatagtcatactgtgtttttttaatgtgttctttgattggaatttagaaggaatggtagaaaaaacattttaaagtaaatcatacatcatgtagatgacaagttagcttaggaatgccttttgtctttcatgctaatatgtaagcattaagcaggtaaagtgttatgcatatacactagtggtgctcgctgatactgtattctctcacactgaggactcccacataagctacataaccatgactactcattggcacttgttccccaaagtcttctgcttcttggcagaaaagtcccagtcattcttctttaagctttagagtaattgtagtaatctctttcatctttaatctctctggtctgagcttaaaatcagtttcctgtttycaaactcataccttaacaattcttagatacctaaaccactacagtcagttttgtttaactgaaggcaaaacaatgtgacatttatttttaaatactagattcctcttatcgtatatattttattaagtatttattttaactactgatctttcataaaaattttaaacttttagtttaaattgctagttcccttgagttaactaatctctatgtcttagaaaccatccttttctaatgttttctaagaaatcttttataaataggtcataagaaacatgttctgcattaaagacccattccttttagcttcccagaggacctgacatagttgttttcctttttttttttcctttttttttttttttttaagataatttaactccagcactaaagccaatgggtgttagttcttgacccaagagatttgttaaccatcaaagcagtatcttgtttcagttcttagrgkgtwtytttgtgacttcagatctagagtggagaatacctcatgtactataacttgaagttattgttctctctgtttatctctagaaaagtttttgctgataatcactacaatttaaattctaagtggaggacagtgcagtaagtaagtcaaaggaaaccttttatgatagcacacctaataaaagtttgaagactattttagtaaatttatatagctagtcaagtgatgaggataaatattaatatccaataatttttatacaagaaatcttttattagcactcataattatgcctgtcacaaagaaagtactcatacttgaattttaatttatttaaatcaaattcatcttgaatatctattattaattattaccagtcagtgatacccaacttcatttcgtagtagaatcatatgggagctttcctgaaattgagtcatgcttcagtctttccaaaacctcttccagtgagtctcactgtagttagagttcagggcaactgctctaaattagttcagtaaaggtccttttctctcctctgttaaatttgtaaattagtaatacaacccaattaagttttgatggcttaaatcccactaaggaatatatcctattgaaaatataaatagtaaattctatcaaccttgcttatggtcaaattaatatttcttaagtgcatgaaattgtattggaacgattcagatgagaagctatagtaaatatctccacacccccagctgcctcagctcttgttttgaagaagtgagaacagaaacactttaagcccaggttactatcttacgatttaacactttggatatatgaattttaaaaaaaaaaagacatatcacaggattgtgtgcagataaaaagatacaggtttaattttcaacatttaaaaaatctacttgtcatgacttctagcaaagctttgacaatcttcaaggagccatgttacatttccccttcactactcctcagaaagtggagttaaattctaaattgctactacagtttttgtttccctattaacatttgaaattgggaattttcaaattttcctagtgagtagtcttactcaccaaccctcctcttgaggctaggccttgtagtgttgcccatgcaggtcttgaactcaaaggctcaagtgatcctgtcgcagctccctgtgtgcctgggagtaccggcatgtgcatcctcgcccagtcccagggtgtgattttccgacaccagtgacgtcaagaataaaataactagatatatcattttgtttaaccttttgtgtatactcttatgtatttgatgtgtgaattacacagttctaataaaacatgcctttctttaaaaaaaaaaaaaaaacttcatgttatagaatgcttcaaagatgctttaatgaaaactattaagaatatatagatttgtatgtcgatttatacttcaaaaatccatatatttgtcatatttatttttttatttgcatggccaaatgatatttaaaatgagtccgtcccccccccccaaatatctggtaactgttaaaactcttgtcacctatccatattcagggttccttaaaaagtagtttaaaattgtatgcatttttatattactatgctgtttgtgtgtatcacatttctaaatattcattattaaattgttactttttaaaactttc

SEQ ID NO: 6 - protein, 920 aa, rattus sp.
MMEGKMADINFKEVTLIVGVVAACYWNSLFCGFVFDDVSAILDTKDLHPSTPLKTLFQNDFWGTPMSEERSHKSYRPLTVLTFRLNYLLSELKPMSYHLLNTVFHAVVSVIFLKVCRLFLDKRGSVIAALLFAVHPIHTEAVTGVVGRAELLSSVFFLAAFLSYTKSKGPDNSIVWTPIALTVLLVAVATLCKEQGITVVGICCVYEVFVAQGYTLPLLCAVAGQFLRGKGSIPFSMLQTLIKLIVLMFSTLLLVVIRVQVIQSQLPVFTRFDNPAAVSPTPTRQLTFNYLLPVNAWLLLNPSELCCDWTMGTIPLIESFLDVRNLATFAFFCFLGALGIFSLRYPGDSSKTVLMALSLMALPFIPASNLFFPVGFVVAERVLYVPSMGFCILVAHGWQKISNKSVLKKLSWVCLSMVILTHALKTLHRNWDWESEYTLFMSALKVNKNNAKLWNNVGHALENEKNFEKALKYFLQATHVQPDDIGAHMNVGRTYKNLNRTKEAEESYMLAKSLMPQIIPGKKYAARIAPNHLNVYINLANLIRANESRLEEADQLYRQAISMRPDFKQAYISRGELLLKMNKPLKAKEAYLKALELDRNNADLWYNLAIVYIELKEPDEALKNFNRALELNSRHKLALFNSAILMQESGEVKLRPEARKRLLNYINEEPQDANGYFNLGMLAMDDKKDSEAETWMKRAIKLQPDFRSALFNLALLYSQTAKELKALPILEELLRYYPDHTKGLILKGDILMNQKKDIAGAKKCFEKILEMDPSNVQGKHNLCVVYFEEKDLLKAERCLVETLALAPHEEYIQRHLSIVRDKIASSGIVDQPLSPVDKTSGTEEKDEVPSEDVKEIISESRPAQIIKTNDNRKSQSNKQSTGNADQDAPHKTTKDIKEIEKKRVAALKRLEEIERILNGE

SEQ ID NO: 7 - DNA, 3832 bp, rattus sp.
gaagatccatttactgatgagcataaggagagacaagaagtggaaatgttggctaagaagtttgaaatgaaatacggtgggaaagcccgtaaacaccggaaggatcggctgcaggatttaattgatataggctttggctacgatgagacagacccgtttattgataactcagaggcttatgacgaattagtccctgcttctctaacaacaaaatatggaggcttctatatcaacactggcactctgcagtttcgccaagcttcagatactgaagaagatgattttacagataaccagaagcacaagccacccaaggtttccaaaataaaagatgatgatattgaggcaaagaagcggaagcggaaagaggaaggggaaaaagaaaagaagccaaggaaaaaagtacccaaacaactgggagttgtggctctcaattcacacaagtctgaaaaaaagaaaaaacgatataaagattccctttctttggctgccatgatacgaaaattccaaaaagagaaggatgccttgaagaaggagtctacccctaaagtcccagtgatcccatcaacttcctctctgcctaagcccccttgtgctgccacaaccctgggggatgatatcccggacttaagtctgaacagtgctgatcctgacctccctatctttgttagcacaaatgagcatgaactatttcaggaagctgaaaatgccctagagatgctggatgattttgactttgacagattactggatgctacttctgatggaagtcccctctctgagtcagggggagaaaatggaaacaccacccagcccaccttcgcctctcaggttgtgcccaaggtggttcctacacttccagatggtctacctgtgcttctggagaaacgcattgaggaccttcgtgtagtaagtgctgccaaactttttgatgaagaaggaaggaaaaaattctttacacaagatatgaataatattcttctagacatcgagttacagctacaagaactaggtcctgtcatccgtagtggtgtctactcccatcttgaagcatttgtgccatgcaataaggaaacactggtaaaacgtctaaagaagttacatcttaacgtccaggatgatcgtttaagagaacctctgcagaaactgaagctggctgttagcaatgtcatgcctgaacagctgtttaaataccaggaggactgccaggctcgcagtcaagccaaatgtgccaagttgcaagcagatgaagaacgagaaaaaaatggatccgatgatgatgatgatgagaaaccagggaagcgagtcataggcccacgaaagaagttccactgggatgacaccatcagaactttgttatgtaaccttgttgaaatcaaattgggatgctatgaactagagccaaataaaagccagtctgctgaggattatcttaaatccttcatggagacagaagtgaagccactgtggcctaagggctggatgcaggcaagaatgctttttaaggaaagccggagtgtacataatcatcttacttctgctccggcaaagaaaaaggtgattcctgcatcaaagcccaaagtcaaggaatgtagccccaaaaaagaccccaaagcgcctgcatccgtggtggcttcaggtggttgtccttgcacgagttccagcacatcgatcgttgcctcagccagctctagctctacaccagcccaagaaaccatctgcctggatgactccctagatgaagacctttctctcccctcagcctctctggatcttgtatctgaagctttagctgtcatcaacaatgggaacaagggcccctcagtcagttcaaggctaaatgtgccaaccacaaaacctcgtccaggcctgagagaagaaaagctagcaagtatcatgagtaagttaccactggctactcccaaaaaactagattctcctcagactgctcactcatcaagtctcattgctggtcacacggggccagtaccaaagaaaccccaggacttagctcacactggtatttcttcaggccttattgctggttcttcaattcagaaccctaaagtttccttagaacctttgccagccaggctgctccagcaaggactgcaaaggtcaagccagatacatgcttcctcttcacagactcatgtctcctcctcccaagcccaagctgctgcctcctctcatgctctgggaacatcagaggctcaagatgcttcttcgttaacacaagtaacaaaggtgcaccagcactcagctgtccagcagaactacgtgtctcctttacaagctaccattagtaaatcacagaccaacccagtggtgaaattaagtaataacccccaactttcctgttcgtcccagttactcaagacttcagagaagccactgatgtatcgcctccctttgtctaccccatcacctggaaatggttctcaggggtctcaccccctggtttctaggacagcaccgagtaccactacctccagtaactatttagccaaggctatggtgtcacaaatctccacgcagggtttcaaatctcccttctcaatggccgcatctcccaaacttgccgcatctcccaaacctgccacatctcctaaacctttgacctcacctaagccttctgtttcacccaagccctctctatcagctaagccttcagtatctactaaactgatttctaaatccaacccaactcccaagcctgctgtatgcccgagttcttctagtccaaacacactagtagcccagagtagccactccacaagtaacaaccctgcccataaacagcccagtggaatgaacatcagcagacagtctcccactctaaatttgttgccctcaaatcgcacttctggccttccgactacaaaaactcttcaggccccttctaaactaacaaactcatcgtccgctggaactgttgggaagaacagcttgagtggaatcccagtgaatgtacctgccagcagaggtagcaaccttaactcaagtggagctaataggactagtctatctgggggaacaggaagtggaacacagggtgctactaaaccgttgtctactccacatagaccaacctctgcctcagggtcttcagtggtaacagccagtgtgcagtctacagcaggagcatcattattggctaatgcctcacctctgactctcatgacatcacctttgtctgtaacaaatcaaactgtgactccctttgggatgctgggtggccttgttccagtgaccatgcccttccagtttcccttggagcttcttggctttggaacggacacagctggagtgacagccacctcgggatctacctcagcggctctccatcatggcctaactcagaatttactaaagagtttacagccaggaactcagcacgcagcaacccttccccactcacctctgcctacacacttacagcaagcatttaatgatggaggccaaagtaaaggggacactaaattaccacggaaacctcagtgactttgcagcaagtgagagaggaaccacgtggctggctagctgggaggtggccgacctgatgggaagtcttggtggtcacagggctgctgttcctgtcgatgtttacattcttgtcccaagcactgtggagagaaggaaaagaaagagtatgttacttgagcaaagccagtgcaggaggaagaaatgcttctgtgcaaagttagtgacctttggtcttttaaaatcaaagttcccatgttaacctacttaaaacagactcagctgtcaaacccacagaagtataaatttgactttatagaaaaaaaaaaaaaaaaaaaaaaaaaaaaaa

SEQ ID NO: 8 - protein, 1167 aa, rattus sp.
EDPFTDEHKERQEVEMLAKKFEMKYGGKARKHRKDRLQDLIDIGFGYDETDPFIDNSEAYDELVPASLTTKYGGFYINTGTLQFRQASDTEEDDFTDNQKHKPPKVSKIKDDDIEAKKRKRKEEGEKEKKPRKKVPKQLGVVALNSHKSEKKKKRYKDSLSLAAMIRKFQKEKDALKKESTPKVPVIPSTSSLPKPPCAATTLGDDIPDLSLNSADPDLPIFVSTNEHELFQEAENALEMLDDFDFDRLLDATSDGSPLSESGGENGNTTQPTFASQVVPKVVPTLPDGLPVLLEKRIEDLRVVSAAKLFDEEGRKKFFTQDMNNILLDIELQLQELGPVIRSGVYSHLEAFVPCNKETLVKRLKKLHLNVQDDRLREPLQKLKLAVSNVMPEQLFKYQEDCQARSQAKCAKLQADEEREKNGSDDDDDEKPGKRVIGPRKKFHWDDTIRTLLCNLVEIKLGCYELEPNKSQSAEDYLKSFMETEVKPLWPKGWMQARMLFKESRSVHNHLTSAPAKKKVIPASKPKVKECSPKKDPKAPASVVASGGCPCTSSSTSIVASASSSSTPAQETICLDDSLDEDLSLPSASLDLVSEALAVINNGNKGPSVSSRLNVPTTKPRPGLREEKLASIMSKLPLATPKKLDSPQTAHSSSLIAGHTGPVPKKPQDLAHTGISSGLIAGSSIQNPKVSLEPLPARLLQQGLQRSSQIHASSSQTHVSSSQAQAAASSHALGTSEAQDASSLTQVTKVHQHSAVQQNYVSPLQATISKSQTNPVVKLSNNPQLSCSSQLLKTSEKPLMYRLPLSTPSPGNGSQGSHPLVSRTAPSTTTSSNYLAKAMVSQISTQGFKSPFSMAASPKLAASPKPATSPKPLTSPKPSVSPKPSLSAKPSVSTKLISKSNPTPKPAVCPSSSSPNTLVAQSSHSTSNNPAHKQPSGMNISRQSPTLNLLPSNRTSGLPTTKTLQAPSKLTNSSSAGTVGKNSLSGIPVNVPASRGSNLNSSGANRTSLSGGTGSGTQGATKPLSTPHRPTSASGSSVVTASVQSTAGASLLANASPLTLMTSPLSVTNQTVTPFGMLGGLVPVTMPFQFPLELLGFGTDTAGVTATSGSTSAALHHGLTQNLLKSLQPGTQHAATLPHSPLPTHLQQAFNDGGQSKGDTKLPRKPQ

SEQ ID NO: 9 - DNA, 3089 bp, rattus sp.
gcagaattgagctgcatcgccttccggagcctccagcgccatgtacgacccagagcgccgctggagcctgtcgttcgcaggctgcggcttcctaggcttctaccacatcggggctacgctatgtctgagcgagcgcgctccgcacatcctccgcgaagcgcgcactttcttcggctgctcggccggtgcactgcacgcggtcaccttcgtgtgcagtctccctctcgatcacatcatggagatcctcatggacctcgtgcggaaagccaggagccgcaacatcggcaccctccacccgttcttcaacattaacaagtgcgtcagagacggccttcaggagaccctcccagacaacgtccaccagatcatttctggcaaggtttacatctcactcaccagagtgtccgatggggagaacgtgctggtgtctgagttccattccaaagacgaagtggtggatgccctggtgtgctcctgcttcattcctctcttctctggcctaatccctccttccttccgaggtgagcggtacgtggatggaggagtgagtgacaacgtccctgtgctggacgccaaaaccaccatcacggtgtcccctttctatggtgagcatgacatctgtcccaaagtgaagtccaccaacttcctccaggtgaatatcaccaacctcagtcttcgtctctgcactgggaaccttcatcttctgaccagagcactcttcccatctgatgtgaaggtgatgggagagctgtgctttcaagggtacctggacgccttccggttcctggaagagaacggcatctgtaatgggccacagcgcagcctgagtctgtccttggagaaggaaatggcgccagaaaccatgataccctgcttggaaaatggccaccttgtagcagggaacaaggtgccagtaagctgtgtatgccttacagctgtgccgtcggatgagagcatctgggagatgctgtcccccaagctcagcacagctctgactgaagcgattaaagacagggggggctacctgaacaaagtctgcaacctcctgcccattaggatcctgtcctacatcttgctgccctgcactctgcccgtggagtcggccatcgctgcagtccacaggctggtgatgtggctccctgatatccatgaagatatccagtggctacagtgggcaacatcccaggtgtgtgcccgaatgaccatgtgcctgctcccctctaccagatccagagcatccaaggataaccatcaaacactcaagcatggatatcacccatctctccacaaaccccaaggcagctctgccggtttgtaaattgctggtctccgtgcttccgatgaacttgggcattctccctgtggatggttccaggagaggccatagctgaaggcactctgccttccaccccaagtccagtttgacctttatctggagcaacagtgtctagatgataggtgggtggggggtgctgtctctctgtttccctctgggaagggttctgttaacttttggaggcagctaggaaatttctctccaggagctgagcctgtgcagctgcccccttggtgctgtgtggtaacctcattgcctgtgaccctaggatcataggatctgggctaaataggtagttcatagaaaccaaagacaataatttggtgtttagaaaactacttttggtctgggtgaagtctggtgcttgagagttagtgcagagagaacagtcaaaccgtctctcagcctgtggatctatggggattccaagggcttcagtgtttggaaacggcaatccaaacgggcaatcttgtgcaatcttggaaggaaaactgttcaggaagtgtgatgggatgagctgtggctgtctctgaaaagggcctaccatataacttattactttcaaggatacctttggctcttactaaaatagtttataaagcattttatagaaacacaccagggaatgcgtggtgaactacatgtatgatcagtgaactgtgactagaattaaccttaaaatctcttgtatgtggggccagagcaacacaggtgggaaacgcagcggacctctgcctcctcggcctcaacatgaacttggcttgctttctccaccgtctccaaatctttgtatagtcatcgaccattaccacctctcctttcccatctactacagcagccttaatggggataagtacccccttttctcaggtgtccgaataagctgtgggtgtggcctgtgtttcctgtaattcagaggttagattggaacataagcaagcagacaaacaagcagacaaacaaacaaggttctactcatattcctaagcagtgacagtgaaggcatgtgtctcccatgcctgagtctcctagggtcctagtgagctctgggttcatgcaagcacttccggaggaattgcaccctccatggaacacataatctccactgggttgatcctgattggataagaaaggatctcggggagagaatgtggttccagaggcaaagtgtctaggctacacagaaaaggtaagactgtccccaagggaagaaaacaaactgggagctggggtccagctcaattgttaagagtgcttctctagtatgcatgaagcccagagtccaatctcagtaccagatacacggtacaggcagtgacatatgcctgtaatcccaaccctcaagcagtagaggcaagaggatcagaagttcatggtcatccttgactacttatacttagggagttggaggtcagccttggctaaatgagaccctgcctctaaaagaaaagcaacaaacaaaaaatagcagaaacttctgccttgctttgaactccccctttctggaagtttcccaccagcagagactattcctgttaccctatcagacaaaactcccactggtttggagtccctccatcctcaggaacaccgggtatcaacagtgaggagcagggagcaatgtcttgactggtaagcccttagcaaagctggttcacttgtttaaaagcaggtgtgaggggttggggatttagctcagtggtagagcgcttgcctaggaagcgcaaggccctgggttcggtccccagctctg

SEQ ID NO: 10 - protein, 425 aa, rattus sp.
MYDPERRWSLSFAGCGFLGFYHIGATLCLSERAPHILREARTFFGCSAGALHAVTFVCSLPLDHIMEILMDLVRKARSRNIGTLHPFFNINKCVRDGLQETLPDNVHQIISGKVYISLTRVSDGENVLVSEFHSKDEVVDALVCSCFIPLFSGLIPPSFRGERYVDGGVSDNVPVLDAKTTITVSPFYGEHDICPKVKSTNFLQVNITNLSLRLCTGNLHLLTRALFPSDVKVMGELCFQGYLDAFRFLEENGICNGPQRSLSLSLEKEMAPETMIPCLENGHLVAGNKVPVSCVCLTAVPSDESIWEMLSPKLSTALTEAIKDRGGYLNKVCNLLPIRILSYILLPCTLPVESAIAAVHRLVMWLPDIHEDIQWLQWATSQVCARMTMCLLPSTRSRASKDNHQTLKHGYHPSLHKPQGSSAGL

SEQ ID NO: 11 - DNA, 7428 bp, rattus sp.
ctggaggactttctgccacctcatgggatgctccagtttgccgatggacaagtcattgcaccaatacacataactataattgatgatactgaatttgaactctctgaaacattcagcatctccttaatgagtgtgactggtggcggccgcctgggcgatgatgttttggtgactgttgtcataccactgaatgactctccctttggaatatttggatttgaagagaagactgtaatggttgatagatcccatttgtctgatgaccctgattcatatgtgaaactgacagttgtccggtccccaggaggaaaaggagctgtccgacttcactggactattgaagagaaagccaaagatgaccttagtcctttgaacgggacactgtattttgatgagactgactcccagaagtccattatactgcacatacttcaaggacaagtgtgctggggaagacaggcgcttcaccattgagctgatggatgctggtgaggtagaaatatctccagggaaaggtagagcatcagtcatcattctagaagagaagagcccatcagaagttggaattgttcatcatccagacacgtcattatcggggagccctcagcaacatacagtggcactgccaccatcagcctggatcgtggcccaggggtttccggggaggtcacagtggactggaggatattgcctccctccaggggggagtttgctgaaacatcaggacaactgaccatgctggatggacagtctactgctactgtagtaattcaggctttggatgatggcatcccagaggagaagtgttcctacgagtttcagctcactggaatcagtgagggtgcggtgctgaatgaagccagcgtcactgccagcatctccatggtggccagtgacgctccctatggccagttctcattttcacatgagcagcttcaagtttccgaagcagcacagaaggttaatgtcacagttgctcgctctggtggttcctttggacgtgtgcgtgtctggtatgagaccggtagcaggacggctgaggcaggctgggactttgttcccacttcaggggagctgatctttgaagcccgagagaagatgaaaagtctgcacattgaaattcttgatgacaaccttcctgaaggccctgaggaattcgttcttgccattacaagagtggacctccagggaagagggtatgatttcaccatccaagagaatggactccagatagatcagcctcctgaaattgggagcatctccgttgttcaaatcataataatggaaaatgacaatgtagaaggcatcattgaatttgacccaaagttcaccgacatctcagtggaggaaggtgctggagtgatcccactccctgtcatgagacttcgagggacttatggccgagtgtcagctgatttcagctcccgggactcctccgctgttccaggaggctatgtactgcatggtggctcagtcacctttcagcacgggcagagcctaagctttataaatgtctccatcgttgatgacaacgacagtgaatcggagaagcagcttgaaattctgctcattggagcgacgggtggagcaatccttgggcgccacctagtgagcaaaatcaccattgccaaaagtggctctccctttggcatcataaggtttctcaaccaaagcaaaatttccgttcctaaccccaattccacaatggctttacacttggtgttagagcggactggcggactcttgggagagattcaggtgagctgggagatagtagggcccagttctgaggaactgttactgccacagcacggagactttgcagacccagtgagtggcacggtctcatttggagatggggaaggtggtgtgagaaccatcaccctcacagtttgtcctcatgaagaaactgaagcggaagagacttttgttgttcagcttaagcctttgaaagaagccaagttagatcccagagctaaagctgttacgctgaccatacagaagtttggggatccaaatggagtcatccattttgctcctgagtctttatctaagaaaatgttctctgagccaccaccgtcagaaggacccatgcttatatccttccttgtcacgagatccaaaggcacctctggagagatcacggtgcactgggagctaagcagtgagtttgaccttacgggagacttcctttccacccaaggatttttcaccattgctgatggagacagcgaagcaagctttgatgtccatttgctgccagatgatgtccctgagatagaggaagaatatgttgtccagctagtttctgtagagggaggtgcagaactggacctggagaaatgcacctcgaggttctctgtatctgccaatgatgacccgcatggcatatttgccctttattcagatcagcagtcagtactgattgggcagaacctcattagatccatccaaattaacatcacccggcttgctggagtgtttggtgctgtggctgtgacacttcaaatactgtctgacaataaggaagacccagttgctactgaaaatgaagagagacaaatggtgatcaaggatggggccagatataaagtaggcttggtgcccttgaaaaatcaggtcttcctatcactgggctccaatttcaccctgcggctgctgtcagcaaggcttctgagtggacccttctatggaatgccaacaattcaccaggaagcaaatcaagcttttctctctgttcctgaggaagctgccaattcccaggttggattcgaatccactgctttccaactcattgacatcacggccggcacaagccaagtcacagtctttaggagaggtacatatggcaggctgtctgtggcctggaccacaggatatgctcctggctcagaaatccctgagcctattgccatcggcaacatgacgccaactctcgggagcctctcttttctgcatggagaacagaagaaaggagtcctcctatggacgtttcccagccctggccagccagaggcctttgtccttcatctgtcaggcctgaggagcagtgctgcaggtggagctcagcttagattggcttttactactgctgaaattgaacccctgggagtcttccagttttccccaggctcaagaaatatcacagtgtcagaggatgcactgataatccgaatatgtgtgcaaagactgtttgggtttcagggtgacctgattaaagtctcctacaagacgactgcaggaagcgctaagcctctggaggacttcgaacctattcagaagggggaaattttttttcagagattccaagctgaggttgattttgaaataaccattattaacgatcagcttcctgagagagaagaaacattttacattaatcttacttcagtagaaactaggggaccaggaaagggagatgtgagttggagacctcgcctaaatccagatctcagtgttgcaatggtcaccatactggacaatgatgacccggctggagtagctgtctctgtccctgtgacagcagggactgtggcagctgacagcactctccttgccatggaagctgattttaccacacaccctaacaaaagcaagataaccaccattccatataccacggaggtgtttgcccctgttacagagacagtggatgtgtctgccatccctgagaaacttgtcaccattcacagcgccatatctgaggagcctgacttggccccaggaactgctcaggctgcagtctttgggacactgagtctcggaccccccattgtctatgtgtcagaggaaatgaagaataacacacccagtactgcagatattcaaatccagaggataggtgggttcgcaggcaatgtcagtgtaacagtgaggacttttgggggaagatgtgctcagaaggaacccagcgtctggccctttcaggatgtttatggaattggcaacctaacatgggcagttgaagaagaagacttcgaagaacaaatgctaaccctgacgtttctcaatggggaaagagaacatagaattgcagttcgaattctggatgatgatgagcccgaggggcaggaattcttctatgtgtttctcactgaccctcaagggggagcagagattgtgaggggaaaggatggctctgggttctcagcctttgctctaatccttatcacaggtagtgacctccacgacggcattgtcggcttcagtgaggagtccctgagaggcctggagctgagggaaggaactgataagagcagtcagcgcctagaggtcacgaggcagcccaacagggcctttgaagaagtccaggtcttttggcgagtcacacttaaccaaacagccaccattctccaggagaaggggctaaacctaacagatgaactccggtctgtggcaggggtcaccacttgcacagtgggtcaaacacagtgttttatccaccttgaactcaatcccaagaaggtgcatcaagttgaaatgcctttctttgtggagctgtatgacgtcactgctggggcagctataaacaacagtgccagatttgcccggattaaagtttccaagagcggggactctcagagccttgttttcttttctgtgggttctcggctggcagtggctcacaagaaggctactttgatcagtttgcaggtggccagagattctgggacaggaatgatgatgtctgttaactttagtacccaggagctgaggagcgctgaaacagttggccggatcctcttatctccagctgtttctgggaaggactttgtgagaacagaaggcacactggtcttcgagcctggccagaaaagtgcggtgttggatgttgtcttaacaccagagacagggtctttaaataaatttcctaaacgcttccagattgtgctttttgacccaaaaggtggtgccagaattgataaagtgtacgggactgccaacatcacccttgtctctgatgtggattctcaagctgtctgggggcttgaaggcctactacatcagcctctacatgaagatgttctcaacagagtgctccatagcctcaacctgagagtagccacagagagcacagatgagcagctcagtgctgtgatgttcataatggaaaagataatgatggaaggaaaaaaccaagctttcagtatagaaagccggactcttttctatgaactcctttgtgctctcattaacccaaagcgcaaagacactaggggattcagccactttgcggaggtggctgagcattttgccttttctcttctgacggatgttacctgtggatcacctggtgaaaaaagcaaaaccatccttgacagttgcccgtatttgtcaatcctggcccttcactggagccctcagcaaatcaatggacacaggtttgaagggaaggaaggtgattacattcaaattccagagaggtttctggatgctcctgagccggaagtgtcggatgggaaaaatgcatgcgcattagtccagtttgtggagtacagcagccagcagtggtttgtagcaggagacaaccttcctgccctgagagacaaggtattgtctttgaatgtgaaaggtcgggatgcacagcccctgcctaacaacaatgaggttctctacaggatttatgcagccgagcctagaattattcctcatacatctctgtgtctcctctggaatcaggctgcttccagytggttgtccgacagccagctttgcaaagtggttgaagatgctgcggactatgtggratgcgcctgctcacacatgtctgcgtatgctgtctacgctcagactgacaacttggtgtcgtacaacgaagcttttttctctgctggctttatatgcatctcaggtctctgtttggctgttgtttcccacgtgttctgtgctagatactccatgtttgcagctaaacttctgactcacatgatggccgccagcttaggagcccaggtttcgttcctggcatctgcatatgcaagtccccagctcagcgaggagagctgttccgccgtggctgctgtggcgcattacctatacctttgccagtttagttggatgcttattcagtctgtgaacttctggtatgtgctggtggtgaatgatgagcacacggagaggcggtgcctgctcttctgccttctgagctgggggcttccatctttcgtggtgattctcctcatagttattttgagaggaatctatcatcagagcatgccgcagatctatggactcattcacggtgacctatgcttcattccaaacatctatgctgctctgttcacggctgctcttgtgcctctaatgtgcctcgtggtggtatttgtggtgttcattcatgcctaccagctgaagccacagtggaaaggttacgatgacgtcttcagaggacgaacaaacgccgcagaaattcctctgattttatatctcttcgccctgatttctctgacgtggctttggggaggactgcatatggcctaccgacacttctggatgttggttctctttgtcattttcaatagtctgcagggactctatgtttttgtggtttatttcattttacacaatcaaacgtgttgccctatgaaggccagctatacggtggaaatgaatggtcacccaggacccagcacagccttcttcactcctgggagtggaatacctccagctggagagatgaacaagtctacccagaacctcatcaatgctatggaagaggtgccatctgactgggagagagtgtccttccaacagaccagccaggcaagccctgatttgaagacaagtccccagaatggtgcctcgtttccttcttctgggggatatggccaagggtccctgatagcagatgaggagtcccaggagtttgatgacctgatatttgcattaaaaactggtgctggtctcagtgtcagtgacaacgagtctggtcaagggagccaagagggaggaaccttgactgattcccggattgtagagctcagaaggataccaattgctgacacccacctctgagagtcccactaacactatccattagaaakaggcaagtttcctcgttgccttggcttttgtactaaactctgtaggcatttacagccatgtagtaatggcctgtatatcataccaagtgagtaacacagaagtgattgttatgtttggaatacacattgctagtgtttttgcaacttaaaaatgactgccacgataaaagatgagatcattcctgtaagttacaaggrtgcacattgtccaaaaatattagtcattttttaatcatccaaactcagctaacattgtttaatgaaaataatcatcaataaatgcattagaatgcagrwrrwaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - protein, 2092 aa, rattus sp.
  UNSURE (1671)...(1671): Xaa=any amino acid
MLDGQSTATVVIQALDDGIPEEKCSYEFQLTGISEGAVLNEASVTASISMVASDAPYGQFSFSHEQLQVSEAAQKVNVTVARSGGSFGRVRVWYETGSRTAEAGWDFVPTSGELIFEAREKMKSLHIEILDDNLPEGPEEFVLAITRVDLQGRGYDFTIQENGLQIDQPPEIGSISVVQIIIMENDNVEGIIEFDPKFTDISVEEGAGVIPLPVMRLRGTYGRVSADFSSRDSSAVPGGYVLHGGSVTFQHGQSLSFINVSIVDDNDSESEKQLEILLIGATGGAILGRHLVSKITIAKSGSPFGIIRFLNQSKISVPNPNSTMALHLVLERTGGLLGEIQVSWEIVGPSSEELLLPQHGDFADPVSGTVSFGDGEGGVRTITLTVCPHEETEAEETFVVQLKPLKEAKLDPRAKAVTLTIQKFGDPNGVIHFAPESLSKKMFSEPPPSEGPMLISFLVTRSKGTSGEITVHWELSSEFDLTGDFLSTQGFFTIADGDSEASFDVHLLPDDVPEIEEEYVVQLVSVEGGAELDLEKCTSRFSVSANDDPHGIFALYSDQQSVLIGQNLIRSIQINITRLAGVFGAVAVTLQILSDNKEDPVATENEERQMVIKDGARYKVGLVPLKNQVFLSLGSNFTLRLLSARLLSGPFYGMPTIHQEANQAFLSVPEEAANSQVGFESTAFQLIDITAGTSQVTVFRRGTYGRLSVAWTTGYAPGSEIPEPIAIGNMTPTLGSLSFLHGEQKKGVLLWTFPSPGQPEAFVLHLSGLRSSAAGGAQLRLAFTTAEIEPLGVFQFSPGSRNITVSEDALIIRICVQRLFGFQGDLIKVSYKTTAGSAKPLEDFEPIQKGEIFFQRFQAEVDFEITIINDQLPEREETFYINLTSVETRGPGKGDVSWRPRLNPDLSVAMVTILDNDDPAGVAVSVPVTAGTVAADSTLLAMEADFTTHPNKSKITTIPYTTEVFAPVTETVDVSAIPEKLVTIHSAISEEPDLAPGTAQAAVFGTLSLGPPIVYVSEEMKNNTPSTADIQIQRIGGFAGNVSVTVRTFGGRCAQKEPSVWPFQDVYGIGNLTWAVEEEDFEEQMLTLTFLNGEREHRIAVRILDDDEPEGQEFFYVFLTDPQGGAEIVRGKDGSGFSAFALILITGSDLHDGIVGFSEESLRGLELREGTDKSSQRLEVTRQPNRAFEEVQVFWRVTLNQTATILQEKGLNLTDELRSVAGVTTCTVGQTQCFIHLELNPKKVHQVEMPFFVELYDVTAGAAINNSARFARIKVSKSGDSQSLVFFSVGSRLAVAHKKATLISLQVARDSGTGMMMSVNFSTQELRSAETVGRILLSPAVSGKDFVRTEGTLVFEPGQKSAVLDVVLTPETGSLNKFPKRFQIVLFDPKGGARIDKVYGTANITLVSDVDSQAVWGLEGLLHQPLHEDVLNRVLHSLNLRVATESTDEQLSAVMFIMEKIMMEGKNQAFSIESRTLFYELLCALINPKRKDTRGFSHFAEVAEHFAFSLLTDVTCGSPGEKSKTILDSCPYLSILALHWSPQQINGHRFEGKEGDYIQIPERFLDAPEPEVSDGKNACALVQFVEYSSQQWFVAGDNLPALRDKVLSLNVKGRDAQPLPNNNEVLYRIYAAEPRIIPHTSLCLLWNQAASSWLSDSQLCKVVEDAADYVXCACSHMSAYAVYAQTDNLVSYNEAFFSAGFICISGLCLAVVSHVFCARYSMFAAKLLTHMMAASLGAQVSFLASAYASPQLSEESCSAVAAVAHYLYLCQFSWMLIQSVNFWYVLVVNDEHTERRCLLFCLLSWGLPSFVVILLIVILRGIYHQSMPQIYGLIHGDLCFIPNIYAALFTAALVPLMCLVVVFVVFIHAYQLKPQWKGYDDVFRGRTNAAEIPLILYLFALISLTWLWGGLHMAYRHFWMLVLFVIFNSLQGLYVFVVYFILHNQTCCPMKASYTVEMNGHPGPSTAFFTPGSGIPPAGEMNKSTQNLINAMEEVPSDWERVSFQQTSQASPDLKTSPQNGASFPSSGGYGQGSLIADEESQEFDDLIFALKTGAGLSVSDNESGQGSQEGGTLTDSRIVELRRIPIADTHL

SEQ ID NO: 13 - DNA, 4097 bp, rattus sp.
attttggtcaacaccctgcttactgcgcacaaccaatcctgttagaactcagcatcccggctcatctgagcagatcctctagtgatttctgctggtcaaaattttttaagataaattgaaactatagcttaattttttttatttatattttttctttttgtatttttttctgcacaaagaaggcggattttttttttcacttattcatgccaagaccaatttttgaaagccagcattgggtgtgtaggatttaagcctatagatagccttgttggaataaaggaaaaggaagatcaaaggagagcacgctggtggaggacggcgccggctaaccttgctaaccattaggaattgctattaagaaaagagagatacaattgtttgttttgagcagtttgttctataagctgactcatcatgtctgccctgacgcctccgactgatatgccaacccccaccactgacaagataacccaggctgccatggagaccatctacctctgcaaattccgggtgtctatggatggagaatggctctgccttcgagagctggatgacatctcccttactgacccagagcctacccatgaagatcctaattatctcatggctaacgagcgcatgaacctgatgaacatggcgaagctgagcatcaagggcttgattgagtcggctctgaacctggggcggaccctggactctgactacgcacctctccagcagttcttcgtggtgatggaacactgcctgaaacatggcttgaaagccaagaaaacttttcttggacaaaataagtccttctggggtcctctagagctggtagaaaagcttgttccagaagctgcagagataacagcaagtgtaaaagatctcccaggactcaagacaccagttggcagaggaagagcctggcttcggttggcattaatgcaaaagaagctttctgagtacatgaaggccttgatcaataagaaggaacttctcagtgagttctatgaagccaatgctctcatgatggaagaagaaggcgcaattattgctggcctcctggtcggtctgaatgtcatcgatgccaatttctgcatgaaaggagaagacctggactctcaggttggcgttatagacttttcaatgtatctcaaagatgggaacagcagtaaaggcagtgaaggggatggacagattactgcgattctggaccagaaaaactatgtagaagaactcaacagacatctgaatgctactgtaaacaaccttcaggcaaaagtagatgcgttagaaaaatccaacacgaaattgacagaggaacttgccgtcgccaacaacagaattattaccttacaagaagaaatggaacgggttaaagaagaaagttcctatctactggaatccaatcggaagggtcctaagcaagacagaactgcagaagggcaagcgctgagcgaagccagaaagcatctaaaggaggagacacagttacgattggacgttgaaaaggagctggagctgcagatcagcatgaggcaggagatggaactggctatgaagatgctggagaaggatgtctgtgagaagcaggatgccctggtgtccctgcggcagcagctggatgatctccgagctcttaaacacgagcttgcctttaagctgcagagttcagacctaggagtgaaacagaaaagtgaattaaacagtcgcttggaagagaagaccaatcagatggctgccaccattaaacagctggagcaaagtgaaaaagatttggtgaaacaggcaaagaccttaaatagtgcagcaaataaactgatcccaaaacatcattaaacgttttgcagttggtcacctcacgagtctgatgtcgcagattaactataaaaggaagaaatctgaaattcctacatttaagctccgattctatataaaatatcacgagacgttgaccttgaatttgttggacttaaaagcactaggtggatatataggttgggatatataggttgggttttgttgttgtggttgccgctgttattttgcccccagagttagaaattttggtatggattcctcattaagtaccaaataaactttgagaaatatttctgagttgaacagtaaatcaaaagaccattttaggaagcccgggctatggaagcactgtgtttgtgtgcttggtagtggtcatactctgtaggtgtggctccctccgcaccagcgagcgagcctgccctgtgcaggggtcacctggcgaccaggaactagtctaacatagctctgcctctcaggccatgacccctctcttctcttgcctcctgtaggtttaacccccagcccaagataaatatttagtatttccttacagttctgttctgagtaaccgaggctgcttgcaatttatttttgccttcgtgatgttggacaaaaagaaaaacagcatccttcctttctttaaagtccttagaaacaaagtagtaattaggtagctcttaacatcacagtgtggaagctggagcaacccctcttaacactgtcatcttctacctctgcccttctctgtcatcctgcttactacttaggtagctcagagagtacaatgctcacgtccgccctgcgtttcctgctggagtttgtgaggccacagtgtagtcatgagttgcaactccctgcctgttgtcttcgcctggaacacttgcccaagagcttcaaacactttttctctaatgagaatttgctaaaggttctgggaaactaatctagttactaaaaaataaacatttctaaatgagaaataaataagatactagcattattgtttaatatcagaatgaagctcttcagtagccacaggcagtgtgagcaggaccttctctaagtgaccgcagatatcacccttttaggctctacatcgtaaataactctatacatactttgatttaaaaaaagactattttggggaggcaggcttttatagacaggagcctttactccatcccgcggtattcacacaatacccagggtttggttccatgttttctgtctccccttctggcaggctatgtagcatagcctgtgctaagagagaggcaaagataatccctttgcttccagctcttcaaaatctttaccttcatggcaaaccagaattaagagtacagcagagttcatgtttctatgggtcctcaaaaggccggttagaggcaacttcatggacagtcctgtgcccctgaactcactttaattcttacagctgtgcacctgcataagcaaagctcctactgcattctgctgttcatcagcctttcacggtgaggagttgccttttacttaagcgtctaaaaagaataatcaaatcaataaaagttagactaaacatagcatgcctctcaatcctactatagaaagcctcgagtatttcaccttctaaaagtgattctttataggtacaaagttataagacattcagaagggagcatccacgagaatcttccagagaacatggtcaggagcttaggaaccccaatgctgactggtcagctgggtcacaaaccacctgataggaatggccgggaaagagccaggcactcgccaggcgcttgctcagagtgtgaccttgtggaaggtgaagtctgggtaagggttgctatggaatgtcctccagcgtcctgcagcacaaggaggaccaggaaatcgtgagaagcccatgtgagaaggagctgctcccgcgtgctcccgctgtgtgtgaaaggcagagtgttaggaaagaaaggcagcggcttcgacgtcaattgctctgtacttgagggtggctctggaaatggatttccaaacagtgatgtcttgttgttgatcatctaatggctgtaaactgtagtaatccagattgcgccaggctgagcgaggccgccagtctgccgagctggacaaccggctcttcaaacaggacttcggagacaagatcaacagcctgcagctggaggtggaggcgctcaccaggcagcggtgagcaggggcagcttggctggcagggtgctgtggtgggcactgcagacctaaacagcaaggagggagtcaggtcctgagtcatcttggtttgttcttgc

SEQ ID NO: 14 - protein, 468 aa, rattus sp.
MSALTPPTDMPTPTTDKITQAAMETIYLCKFRVSMDGEWLCLRELDDISLTDPEPTHEDPNYLMANERMNLMNMAKLSIKGLIESALNLGRTLDSDYAPLQQFFVVMEHCLKHGLKAKKTFLGQNKSFWGPLELVEKLVPEAAEITASVKDLPGLKTPVGRGRAWLRLALMQKKLSEYMKALINKKELLSEFYEANALMMEEEGAIIAGLLVGLNVIDANFCMKGEDLDSQVGVIDFSMYLKDGNSSKGSEGDGQITAILDQKNYVEELNRHLNATVNNLQAKVDALEKSNTKLTEELAVANNRIITLQEEMERVKEESSYLLESNRKGPKQDRTAEGQALSEARKHLKEETQLRLDVEKELELQISMRQEMELAMKMLEKDVCEKQDALVSLRQQLDDLRALKHELAFKLQSSDLGVKQKSELNSRLEEKTNQMAATIKQLEQSEKDLVKQAKTLNSAANKLIPKHH

SEQ ID NO: 15 - DNA, 18946 bp, human
atgtctgatctcaaagaagggatgccctctgcatctttattagtaaatcttctttcagctttactcatcctatttgtgtttggagaaacagaaataagatttactggacaaactgaatttgttgttaatgaaacaagtacaacagttattcgtcttatcattgaaaggataggagagccagcaaatgttactgcaattgtatcgctgtatggagaggacgctggtgacttttttgacacatatgctgcagcttttatacctgccggagaaacaaacagaacagtgtacatagcagtatgtgatgatgacttaccagagcctgacgaaacttttatttttcacttaacattacagaaaccttcagcaaatgtgaagcttggatggccaaggactgttactgtgacaatattatcaaatgacaatgcatttggaattatttcatttaatatgcttccctcaatcgcagtgagtgagcccaagggcagaaatgagtctatgcctcttactctcatcagggaaaagggaacctatggaatggtcatggtgacttttgaggtagagggtggcccaaatccccctgatgaagatttgagtccagttaaaggaaatatcacctttccccctggcagagcaacagtaatttataacttgacagtactcgatgacgaggtaccagaaaatgatgaaatatttttaattcaactgaaaagtgtagaaggaggagctgagattaacacctctaggaattccattgagatcatcattaagaaaaatgatagtcccgtgagattccttcagagtatttatttggttcctgaggaagaccacatactcataattccagtagttcgtggaaaggacaacaatggaaatctgattggatctgatgaatatgaggtttcaatcagttatgctgtcacaactgggaattccacagcacatgcccagcaaaatctggacttcattgatcttcagccaaacacaactgttgtttttccaccttttattcatgaatctcacttgaaatttcaaatagttgatgacaccataccggagattgctgaatcgtttcacattatgttactaaaagataccttacagggagatgctgtgctaataagcccttctgttgtacaagtcaccattaagccaaatgataaaccttatggagtcctttcattcaacagtgttttgtttgaaaggacagttataattgatgaagatagaatatcaagatatgaagaaatcacagtggttagaaatggaggaacccatgggaatgtctctgcgaattgggtgttgacacggaacagcactgatccctcaccagtaacagcagatatcagaccgagctctggagttctccattttgcacaagggcagatgttggcaacaattcctcttactgtggttgatgatgatcttccagaagaggcagaagcttatctacttcaaattctgcctcatacaatacgaggaggtgcagaagtgagcgagccagcggagcttttgttctacattcaggatagtgatgatgtctatggcctaataacattttttcctatggaaaaccagaagattgaaagcagcccatgtgaacgatacttatccttgagttttacaagactaggagggactaaaggagatgtgaggttgctttattctgtactttacattcctgctggagctgtggaccccttgcaagcaaaagaaggcatcttaaatatatcaaggagaaatgacctcatttttccagagcaaaaaactcaagtcactacaaaattaccaataagaaatgatgcattccttcaaaatggagctcactttctagtacagttggaaactgtggagttgttaaacataattcctctaatcccacccataagccctagatttggggaaatctgcaatatttctttactggttactccagccattgcaaatggagaaattggctttctcagcaatcttccaattattttgcatgaaccagaagattttgctgctgaagtggtatacattcccttacatcgggatggaactgatggccaggctactgtctactggagtttgaagccctctggctttaattcaaaagcagtgaccccggatgatataggcccctttaatggctctgttttgtttttatctgggcaaagtgacacaacaatcaacattactatcaaaggtgatgacataccggaaatgaatgaaactgtaacactttctctagacagggttaacgtggaaaaccaagtgctgaaatctggatatactagccgtgacctaattattttggaaaatgatgaccctgggggagtttttgaattttctcctgcttccagaggaccctatgttataaaagaaggagaatctgtagagctccacatcatccgatcaagggggtcccttgttaagcagtttctacactaccgagtagagccaagagatagcaatgaattctatggaaacacgggagtactagaatttaaacctggagaaagggagatagtgatcaccttgctagcaagattggatgggataccagagttggatgaacactactgggtggtcctcagcagccacggagaacgggaaagcaagttgggaagtgccaccattgtcaatataacgattctgaaaaatgatgatcctcatggcattatagaatttgtttctgatggtctaattgtgatgataaatgaaagcaaaggagatgctatctatagtgctgtttatgatgtagtaagaaatcgaggcaactttggtgatgttagtgtatcatgggtggttagtccagactttacacaagatgtatttcctgtacaagggactgttgtctttggagatcaggaattttcaaaaaatatcaccatttactcccttccagatgagattccagaagaaatggaagaatttaccgttatcctactgaatggcactggaggagctaaagtgggaaatagaacaactgcaactctgaggattagaagaattgatgaccccatttattttgcagaacctcgtgtagtgagggttcaggaaggtgagactgccaactttacagttctcagaaatggatctgttgatgtgacttgcatggtccagtatgctaccaaggatgggaaggctactgcaagagagagagatttcattcctgttgaaaaaggagaaacgctcatttttgaggttggaagtagacagcagagcatatccatatttgttaatgaagatggtatcccggaaacagatgagcccttttatataatcctcttgaattcaacaggtgatacagtagtatatcaatatggagtagctacagtaataattgaagctaatgatgacccaaatggcattttttctctggagcccatagacaaagcagtggaagaaggaaagactaatgcattttggattttgaggcaccgaggatactttggtagtgtttctgtatcttggcagctctttcagaatgattctgctttgcagcctgggcaggagttctatgaaacttcaggaactgttaacttcatggatggagaagaagcaaaaccaatcattctccatgcttttccagataaaattcctgaattcaatgaattttatttcctaaaacttgtaaacatttcaggtggatccccaggtcctgggggccagctagcagaaaccaacctccaggtgacagtaatggttccattcaatgatgatccctttggagtttttatcttggatccagagtgtttagagagagaagtggcagaagatgtcctgtctgaagatgatatgtcttatattaccaacttcaccattttgaggcagcagggtgtgtttggtgatgtacaactgggctgggaaatactgtccagtgagttccctgctggtttgccaccaatgatagattttttactggttggaattttccccaccaccgtgcatttacaacagcacatgcggcgtcaccacagtggaacggatgctttgtactttaccggactagagggtgcatttgggactgttaatccaaaataccatccctccaggaataatacaattgccaactttacattctcagcttgggtaatgcccaatgccaatacgaatggattcattatagcgaaggatgacggtaatggaagcatctactacggggtaaaaatacaaacaaacgaatcccatgtgacactttcccttcattataaaaccttgggttccaatgctacatacattgccaagacaacagtcatgaaatatttagaagaaagtgtttggcttcatctactaattatcctggaggatggtataatcgaattctacctggatggaaatgcaatgcccaggggaatcaagagtctgaaaggagaagccattactgacggtcctgggatactgagaattggagcagggataaatggcaatgacagatttacaggtctgatgcaggatgtgaggtcctatgagcggaaactgacgcttgaagaaatttatgaacttcatgccatgcccgcaaaaagtgatttacacccaatttctggatatctggagttcagacagggagaaactaacaaatcattcattatttctgcaagagatgacaatgacgaggaaggagaagaattattcattcttaaactagtttctgtatatggaggagctcgtatttcggaagaaaatactactgcaagattaacaatacaaaaaagtgacaatgcaaatggcttgtttggtttcacaggagcttgtataccagagattgcagaggagggatcaaccatttcttgtgtggttgagagaaccagaggagctctggattatgtgcatgttttttacaccatttcacagattgaaactgatggcattaattaccttgttgatgactttgctaatgccagtggaactattacattccttccttggcagagatcagaggttctgaatatatatgttcttgatgatgatattcctgaacttaatgagtatttccgtgtgacattggtttctgcaattcctggagatgggaagctaggctcaactcctaccagtggtgcaagcatagatcctgaaaaggaaacgactgatatcaccatcaaagctagtgatcatccatatggcttgctgcagttctccacagggctgcctcctcagcctaaggacgcaatgaccctgcctgcaagcagcgttccacatatcactgtggaggaggaagatggagaaatcaggttattggtcatccgtgcacagggacttctgggaagggtgactgcggaatttagaacagtgtccttgacagcattcagtcctgaggattaccagaatgttgctggcacattagaatttcaaccaggagaaagatataaatacattttcataaacatcactgataattctattcctgaactggaaaaatcttttaaagttgagttgttaaacttggaaggaggggctgaactctttagggttgatggaagtggtagtggtgatggggacatggaattcttccttccaactattcacaaacgtgccagtctaggagtggcttcccaaattctagtgacaattgcagcctctgaccacgctcatggcgtatttgaatttagccctgagtcactctttgtcagtggaactgaaccagaagatgggtatagcactgttacattaaatgttataagacatcatggaactctgtctccagtgactttgcattggaacatagactctgatcctgatggtgatctcgccttcacctctggcaacatcacatttgagattgggcagatgagcgccaatatcactgtggagatattgtctgacgaagacccagaactggataaggcattctctgtgtcaatcctcagtgtttccagtggttctttgggagctcatattaatgccacgttaacagttttggctagtgatgatccatatgggatattcattttttctgagaaaaacagacctgttaaagttgaggaagcaacccagaacatcacactatcaataataaggttgaaaggcctcatgggaaaagtccttgtctcatatgcaacactagatgatatggaaaaaccaccttattttccacctaatttagcgagagcaactcaaggaagagactatataccagcttctggatttgctctttttggagctaatcagagtgaggcaacaatagctatttcaattttggatgatgatgagccagaaaggtccgaatctgtctttatcgaactactcaactctactttagtagcgaaagtacagagtcgttcaattccaaattctccacgtcttgggcctaaggtagaaactattgcgcaactaattatcattgccaatgatgatgcatttggaactcttcagctctcagcaccaattgtccgagtggcagaaaatcatgttggacccattatcaatgtgactagaacaggaggagcatttgcagatgtctctgtgaagtttaaagctgtgccaataactgcaatagctggtgaagattatagtatagcttcatcagatgtggtcttgctagaaggggaaaccagtaaagccgtgccaatatatgtcattaatgatatctatcctgaactggaagaatcttttcttgtgcaactgatgaatgaaacaacaggaggagccagactaggggctttaacagaggcagtcattattattgaggcctctgatgacccctatggattatttggttttcagattactaaacttattgtagaggaacctgagtttaactcagtgaagataaacctgccaataattcgaaattctgggacactcggcaatgttactgttcagtgggttgccaccattaatggacagcttgctactggcgacctgcgagttgtctcaggtaatgtgacctttgcccctggggaaaccattcaaaccttgttgttagaggtcctggctgacgacgttccggagattgaagaggttatccaagtgcaactaactgatgcctctggtggaggtactattgggttagatcgaattgcaaatattattattcctgccaatgatgatccttatggtacagtagcctttgctcagatggtttatcgtgttcaagagcctctggaaagaagttcctgtgctaatataactgtcaggcgaagcggagggcactttggtcggctgttgttgttctacagtacttccgacattgatgtagtggctctggcaatggaggaaggtcaagatttactgtcctactatgaatctccaattcaaggggtgcctgacccactttggagaacttggatgaatgtctctgccgtgggggagcccctgtatacctgtgccactttgtgccttaaggaacaagcttgctcagcgttttcatttttcagtgcttctgagggtccccagcgtttctggatgacatcatggatcagcccagctgtcaacaattcagacttctggacctacaggaaaaacatgaccagggtagcatctctttttagtggtcaggctgtggctgggagtgactatgagcctgtgacaaggcaatgggccataatgcaggaaggtgatgaattcgcaaatctcacagtgtctattcttcctgatgatttcccagagatggatgagagttttctaatttctctccttgaagttcacctcatgaacatttcagccagtttgaaaaatcagccaaccataggacagccaaatatttctacagttgtcatagcactaaatggtgatgcctttggagtgtttgtgatctacagtattagtcccaatacttccgaagatggcttatttgttgaagttcaggagcagccccaaaccttggtggagctgatgatacacaggacagggggcagcttaggtcaagtggcagtcgaatggcgtgttgttggtggaacagctactgaaggtttagattttataggtgctggagagattctgacctttgctgaaggtgaaaccaaaaagacagtcattttaaccatcttggatgactctgaaccagaggatgacgaaagtatcatagttagtttggtgtacactgaaggtggaagtagaattttgccaagctccgacactgttagagtgaacattttggccaatgacaatgtggcaggaattgttagctttcagacagcttccagatctgtcataggtcatgaaggagaaattttacaattccatgtgataagaactttccctggtcgaggaaatgttactgttaactggaaaattattgggcaaaatctagaactcaattttgctaactttagcggacaacttttctttcctgaggggtcgttgaatacaacattgtttgtgcatttgttggatgacaacattcctgaggagaaagaagtataccaagtcattctgtatgatgtcaggacacaaggagttccaccagccggaatcgccctgcttgatgctcaaggatatgcagctgtcctcacagtagaagccagtgatgaaccacatggagttttaaattttgctctttcatcaagatttgtgttactacaagaggctaacataacaattcagcttttcatcaacagagaatttggatctctaggagctatcaatgtcacatataccacggttcctggaatgctgagtctgaagaaccaaacagtaggaaacctagcagagccagaagttgattttgtccctatcattggctttctgattttagaagaaggggaaacagcagcagccatcaacattaccattcttgaggatgatgtaccagagctagaagaatatttcctggtgaatttaacttacgttggacttaccatggctgcttcaacttcatttcctcccagactagattcagaaggtttgactgcacaagttattattgatgccaatgatggggcccgaggtgtaattgaatggcaacaaagcaggtttgaagtaaatgaaacccatggaagtttaacattggtagcccagaggagcagagaacctcttggccatgtttccttatttgtgtatgctcagaatttggaagcacaagtggggctggattatatcttcaccccaatgattcttcattttgctgatggagaaaggtataaaaatgtcaatatcatgattcttgatgatgacattccagaaggagatgaaaaatttcagctgattttaacaaatccttctcctggactagagctagggaaaaatacaatagccttaattattgtccttgctaatgatgacggccctggagttctatcatttaacaacagtgagcactttttcctaagagagccaacagctctctacgtccaggagagtgttgcagtattgtacattgttcgggaacctgcacaaggattgtttggaacagtgacagttcagttcattgtgacagaagtgaattcctcaaatgaatctaaagatctgactccttccaaaggctatattgttttagaagaaggtgttcgattcaaggccctacaaatatctgccatattagacacggaaccagaaatggatgagtattttgtttgcaccttgtttaatccaactggaggtgctagactaggggtgcatgttcaaaccctgataacagttttgcaaaaccaggcccctttggggctattcagtatctctgcagttgaaaatagagccacctccatagacatcgaagaagccaataggaccgtgtatttaaatgtatctcgaactaatggcattgatttggctgtgagtgtgcagtgggagacagtatctgaaacagcctttggcatggggggaatggatgttgtgttttccgtatttcaaagttttttggatgaatcagcttctggctggtgtttctttactttggaaaatttaatatatggtataatgttaagaaaatcatctgttactgtttaccgatggcaggggatttttattccagttgaggatttaaatatagaaaatcctaaaacttgtgaggcctttaatattggtttttctccctacttcgtgattactcatgaagaaagaaatgaagaaaagccttctcttaacagtgtgtttacattcacatctggatttaaattattcctggtacaaacaatcattattctggaaagttctcaagtaagatattttacttcagacagccaagattatttaatcattgcaagtcaaagagatgattccgaattaactcaggtcttcaggtggaatggaggaagcttcgtgttgcatcaaaaactccctgtccgaggtgtgctgaccgtggccttgttcaacaagggaggctctgtgttcttagccatttcccaggctaatgccaggctaaactcccttttattcagatggtctggcagtgggtttattaactttcaagaggtgcctgtcagtgggacaacagaagttgaggctttgtcttcagccaatgatatttacctaatatttgccaaaaatgtctttctaggagatcagaattcaattgatattttcatctgggagatgggacagtcttccttcaggtattttcagtctgtagattttgctgctgttaacagaatccactccttcacaccagcctcaggaatagcccacatacttcttattggccaagatatgtctgctctttactgctggaattcggagcgtaatcaattctcttttgttctggaagtaccttctgcttatgatgtggcttctgttacagtaaagtcccttaattcaagcaagaatttaatagctctagtgggagctcattcacatatatatgagctagcctacatttccagccattctgactttattcctagttcaggtgaactgatatttgaacctggtgagagagaagctacaatagcagtaaatatccttgatgatacagttccagaaaaagaagaatccttcaaagttcaacttaaaaatcccaaaggaggagcagagattggcattaatgattctgtaacaataaccattctgtctaatgatgatgcctatggaattgttgcatttgctcagaattcattatataagcaagtggaagaaatggagcaagatagcctagtaaccttgaacgttgaacgcttaaaaggaacatatggccgtataaccatagcatgggaagctgatggaagtattagtgatatatttcctacctcaggagtgattttatttactgaaggccaggtactgtcaacaatcactctaactattcttgctgataatataccagagttatcagaggttgtgattgtaaccctcacccgtatcaccacagaaggggttgaggactcatacaaaggtgctactattgatcaggacagaagcaagtctgttataacaactttgcccaatgactcaccttttggcttggtgggctggcgtgctgcgtctgtcttcattagagtagcagagcctaaagaaaacaccaccactcttcagttacaaatagctcgagataaaggactacttggggatattgccattcacttgagagctcaacccaatttcttactgcatgtcgataatcaagctactgagaatgaagattatgtattgcaagaaacaataataataatgaaagaaaacataaaagaagctcatgccgaagtttccattttgccggatgaccttcctgaattggaggaaggatttattgtcactatcactgaggtgaacctggtgaactctgacttctctacaggacagccaagtgtgcggaggcccggaatggaaatagctgagataatgatagaagaaaatgacgatcccagaggaatttttatgtttcatgttactagaggcgctggggaagttattactgcctatgaggtgcctccacccttgaacgttcttcaagttcctgtagtccggctggctggaagctttggggcagtaaatgtttattggaaagcatcaccagacagtgctggcctggaagactttaaaccatctcatgggattcttgaatttgcagataaacaggttactgcaatgatagaaatcaccataattgatgatgctgaatttgaattgacagagacgttcaatacttccttgatcagtgttgctggaggtggcagacttggtgatgatgttgtggtaactgttgttattccacaaaatgattctccatttggagtatttggatttgaagaaaagactgtaatgattgatgaatccctttcatccgatgaccctgattcatatgtgacattgacggttgtccggtccccaggaggaaaaggaaccgtccgacttgagtggaccatagatgagaaggctaaacataaccttagtcctttgaatgggacccttcattttgatgagactgagtcccagaagaccattgtgttgcacacacttcaagacacagtgttggaggaggacaggcgtttcaccattcagctgatatcaattgatgaggtagaaatatctccagtaaaaggtagtgcatcaataattattcggggtgataagcgagcatcaggagaagttgggatagctccgtcatctaggcacatcctcattggggaaccctcagcaaaatataatggtaccgctattatcagccttgttcgaggcccagggattttgggggaggtcacagtgttctggaggatattccctccttccgtgggggaatttgctgaaacatcaggaaaactgacaatgcgagacgaacagtctgcagtcattgtagtaatacaggctttgaacgatgacattcccgaggaaaaaagcttctatgagtttcagctcactgcagtcagtgagggaggagttctgagtgaatccagcagcactgccaacatcacggtggtggccagcgactctccctatggccgatttgccttttcacatgagcaacttcgagtgtcagaagcacagagggttaacatcacaatcatccgttccagtggagattttggccatgtgcgactctggtacaagacgatgagcgggacagcggaagcaggcttggattttgttcctgcagcaggggagctcctctttgaagcaggggagatgaggaaaagtctgcatgttgaaatccttgatgatgactatcctgaaggcccagaggaattttctctaacaattacaaaggtggaactccagggaagagggtatgattttaccattcaagaaaatggacttcagatagatcaacctcctgaaataggaaacatctccattgttcgcatcataataatgaaaaatgataacgcagaaggcatcattgaatttgacccaaagtatactgccttcgaagtggaggaagatgttgggctgatcatgatcccagtggtgaggctacatggaacttatggctatgtgacagctgatttcatctctcagagctcctctgccagtcccggaggtgttgattacattttgcatggcagtacagtcacctttcagcatgggcaaaacttaagttttataaatatctccatcattgatgacaatgaaagtgaatttgaggagcccattgaaattctactcactggagctactggaggagcggtccttgggcgccacctagtgagcagaatcataatagctaagagtgactctccctttggagttataaggtttctcaatcaaagcaaaatttctattgctaatcccaattccacaatgattttatcactggtgctggagcggactggaggactcttgggagagattcaggtgaactgggagacagtaggacccaactctcaagaagccttactgccacagaatagagacattgcagacccagtgagcgggttgttctattttggagaaggagaaggaggagtgagaaccataattctgacaatctatcctcatgaagaaattgaagttgaagagacattcattattaaacttcatcttgtgaaaggagaagctaaattagactccagagctaaagatgttacattaaccatacaagagtttggtgacccaaatggagttgttcagtttgctcctgaaactttgtctaagaagacttattcagagcctctggctctggaagggcccctgctcattaccttctttgtcagaagagtcaagggcacctttggagagattatggtttactgggaattaagtagtgagtttgacattactgaagactttctttccaccagtggatttttcaccattgctgatggagagagtgaagctagctttgatgttcatttgctaccagatgaggtacctgagatagaggaagattatgtgatccagcttgtttctgtagagggaggagccgaactggatctggagaagagtatcacatggttctctgtttatgcaaatgatgacccacatggagtatttgccctgtattcggatcgccagtcaatacttattgggcagaaccttattagatccatccaaattaacataacccggcttgctggaacatttggagatgtggctgttgggcttcgaatatcatcggatcataaagaacagccgattgttaccgaaaatgcagagaggcagctggtggtcaaagatggtgccacatataaagtggacgtggtgccaataaagaatcaggtcttcctatcactgggctctaatttcactttgcaactggtgactgtgatgcttgtcggtggacgtttctatggaatgccaacaattcttcaggaagcaaaatctgctgtccttccagtctctgagaaagctgccaattctcaggtcggatttgaatccactgcttttcaactcatgaacatcactgctggcacaagccacgttatgatttctaggagaggcacatatggagctctctcggttgcctggaccactggatatgctcctgggttagaaattcctgaattcattgttgttggcaacatgaccccaacactggggagcctttcattttcccacggtgaacaaaggaaaggagttttcctgtggacgtttcctagccctggttggccagaggcctttgttcttcacctatcaggagtgcagagcagtgctcctggcggagctcaactccgatcaggtttcattgttgctgaaattgaaccaatgggcgtcttccaattttccactagctcaagaaatatcatagtgtcagaagatacacagatgatcagattacatgtacaaagactatttgggttccacagcgatcttattaaagtttcttatcagaccactgcaggaagcgccaagccactggaagattttgagcctgttcagaatggggaactgttttttcaaaaattccaaactgaggttgattttgaaataaccattattaatgatcagctttctgagatagaagaatttttttacattaaccttacttcagtagaaattaggggattacaaaagtttgatgttaattggagcccacgcctgaatctagatttcagtgttgcagtgattacaatattggataatgatgacctggcaggaatggatatttccttccccgagacaactgtggctgtagcagttgacacaactctcattcctgtagaaactgaatccaccacatacctcagcacaagcaagacgactaccattctgcagccaaccaacgtggttgccattgttactgaggcaactggtgtatctgccatccctgagaaacttgtcacccttcatggcacacctgctgtgtctgaaaagcctgatgtggccactgtaactgccaatgtttccattcatggaacattcagccttgggccatccattgtttatattgaagaggagatgaagaatggcacattcaacactgcagaagttcttatccgaagaactggtgggtttactggcaatgtcagcataacagttaaaactttcggtgaaagatgtgctcagatggaaccaaatgcattgccctttcgtggtatctatgggatttccaacctaacatgggcagttgaagaagaagactttgaagaacaaactcttacccttatattcctagatggagaaagagaacgtaaagtatcagttcaaattttggatgatgatgagcctgaggggcaggaattcttctacgtgtttctcacaaaccctcaagggggagcacagattgtggaggggaaggatgatactggatttgcagcttttgccatggttattattacagggagtgaccttcacaatggcatcataggattcagtgaggagtcccagagtggactagaactcagggaaggagctgttatgagaagattgcaccttattgtcacaagacagccaaacagggcctttgaagatgtcaaggtcttttggcgagtcacacttaacaaaacagtcgtcgtgctccagaaggatggggtaaacctgatggaggaacttcagtctgtgtcagggaccacaacctgtacaatgggtcaaacaaaatgctttatcagcattgaactcaaaccagaaaaggtaccacaggttgaagtgtatttttttgtggaactatatgaagctactgctggagcagcaataaacaacagtgccagattcgcacagattaaaatcttagaaagtgatgaatctcaaagccttgtgtatttttctgtgggttctcggctggcagtggctcacaagaaggccactttaatcagtctgcaggtggccagagattctgggacaggactaatgatgtctgttaactttagtacccaggagttgaggagtgctgaaacaattggtcgtaccatcatatctccagctatttctggaaaggattttgtgataactgaaggcacattggtctttgaacctggccagagaagcactgtattggatgtcatcctaacgccagagacaggatctttaaattcatttcctaaacgcttccagattgtcctttttgacccaaaaggtggtgccagaattgataaagtgtatgggactgccaacatcactcttgtctcagatgcagattcgcaggccatttgggggcttgcagatcagctacatcagcctgtgaatgatgatattctcaacagagtgctccataccatcagcatgaaagtggccacagaaaacacagatgaacaactcagtgccatgatgcatttaatagaaaagataactactgaaggaaaaattcaagctttcagtgttgccagccgaactcttttctatgagattctttgttctcttattaacccaaagcgcaaggacactaggggattcagtcactttgctgaagtgactgagaattttgccttttctctgctgactaatgttacttgcggctctcctggtgaaaaaagcaaaaccatccttgatagttgcccatatttgtcaatattggctcttcactggtatcctcagcaaatcaatggacacaagtttgaaggaaaggaaggagattacattcgaattccagagaggctactggatgtccaggatgcagaaataatggctgggaaaagtacatgtaaattagtccagtttacagagtatagcagccaacagtggtttataagtggaaacaatcttcctaccctaaaaaataaggtattatctttgagtgtgaaaggtcagagttcacaactcctgactaatgacaatgaggttctctacaggatttatgctgctgagcctagaattattcctcagacatctctgtgtctcctttggaatcaggctgctgcaagctggttgtctgacagtcagttttgcaaagtggttgaggaaactgcagactatgtggaatgtgcctgttcacacatgtctgtgtatgctgtctatgctcggactgacaacttgtcttcatacaatgaagccttcttcacttctggatttatatgtatctcaggtctttgcttggctgttctttcccatatcttctgtgccaggtactccatgtttgcagctaaacttctgactcacatgatggcagccagcttaggtacacagattctgtttctggcgtctgcatacgcaagtccccaactcgctgaggagagctgttcagctatggctgctgtcacacattacctgtatctttgccagtttagctggatgctcattcagtctgtgaatttctggtacgtgctggtgatgaatgatgagcacacagagaggcgatatctgctgtttttccttctgagttggggactaccagcttttgtggtgattctcctcatagttattttgaaaggaatctatcatcagagcatgtcacagatctatggactcattcatggtgacctgtgttttattccaaacgtctatgctgctttgttcactgcagctcttgttcctttgacgtgcctcgtggtggtgttcgtggtgttcatccatgcctaccaggtgaagccacagtggaaagcatatgatgatgtcttcagaggaaggacaaatgctgcagaaattccactgattttatatctctttgctctgatttccgtgacatggctttggggaggactacacatggcctacagacacttctggatgttggttctctttgtcattttcaacagtctgcagggactttatgttttcatggtttatttcattttacacaaccaaatgtgttgccctatgaaggccagttacactgtggaaatgaatgggcatcctggacccagcacagcctttttcacgcccgggagtggaatgcctcctgctggaggggaaatcagcaagtccacccagaatctcatcggtgctatggaggaggtgccacctgactgggagagagcatccttccaacagggcagtcaggccagccctgatttaaagccaagtccacaaaatggagccacgttcccgtcctctggaggatatggccaggggtcactgatagccgatgaggagtcccaggagtttgatgatttaatatttgcattaaaaactggtgctggtctcagtgtcagtgataatgaatctggtcaaggcagccaggaggggggcaccttgactgactcccagatcgtggagctcaggaggatacccatcgccgacactcacctgtagcacctcactaaccattcgactgagcacactt

SEQ ID NO: 16 - protein, 6304 aa, human
MSDLKEGMPSASLLVNLLSALLILFVFGETEIRFTGQTEFVVNETSTTVIRLIIERIGEPANVTAIVSLYGEDAGDFFDTYAAAFIPAGETNRTVYIAVCDDDLPEPDETFIFHLTLQKPSANVKLGWPRTVTVTILSNDNAFGIISFNMLPSIAVSEPKGRNESMPLTLIREKGTYGMVMVTFEVEGGPNPPDEDLSPVKGNITFPPGRATVIYNLTVLDDEVPENDEIFLIQLKSVEGGAEINTSRNSIEIIIKKNDSPVRFLQSIYLVPEEDHILIIPVVRGKDNNGNLIGSDEYEVSISYAVTTGNSTAHAQQNLDFIDLQPNTTVVFPPFIHESHLKFQIVDDTIPEIAESFHIMLLKDTLQGDAVLISPSVVQVTIKPNDKPYGVLSFNSVLFERTVIIDEDRISRYEEITVVRNGGTHGNVSANWVLTRNSTDPSPVTADIRPSSGVLHFAQGQMLATIPLTVVDDDLPEEAEAYLLQILPHTIRGGAEVSEPAELLFYIQDSDDVYGLITFFPMENQKIESSPCERYLSLSFTRLGGTKGDVRLLYSVLYIPAGAVDPLQAKEGILNISRRNDLIFPEQKTQVTTKLPIRNDAFLQNGAHFLVQLETVELLNIIPLIPPISPRFGEICNISLLVTPAIANGEIGFLSNLPIILHEPEDFAAEVVYIPLHRDGTDGQATVYWSLKPSGFNSKAVTPDDIGPFNGSVLFLSGQSDTTINITIKGDDIPEMNETVTLSLDRVNVENQVLKSGYTSRDLIILENDDPGGVFEFSPASRGPYVIKEGESVELHIIRSRGSLVKQFLHYRVEPRDSNEFYGNTGVLEFKPGEREIVITLLARLDGIPELDEHYWVVLSSHGERESKLGSATIVNITILKNDDPHGIIEFVSDGLIVMINESKGDAIYSAVYDVVRNRGNFGDVSVSWVVSPDFTQDVFPVQGTVVFGDQEFSKNITIYSLPDEIPEEMEEFTVILLNGTGGAKVGNRTTATLRIRRIDDPIYFAEPRVVRVQEGETANFTVLRNGSVDVTCMVQYATKDGKATARERDFIPVEKGETLIFEVGSRQQSISIFVNEDGIPETDEPFYIILLNSTGDTVVYQYGVATVIIEANDDPNGIFSLEPIDKAVEEGKTNAFWILRHRGYFGSVSVSWQLFQNDSALQPGQEFYETSGTVNFMDGEEAKPIILHAFPDKIPEFNEFYFLKLVNISGGSPGPGGQLAETNLQVTVMVPFNDDPFGVFILDPECLEREVAEDVLSEDDMSYITNFTILRQQGVFGDVQLGWEILSSEFPAGLPPMIDFLLVGIFPTTVHLQQHMRRHHSGTDALYFTGLEGAFGTVNPKYHPSRNNTIANFTFSAWVMPNANTNGFIIAKDDGNGSIYYGVKIQTNESHVTLSLHYKTLGSNATYIAKTTVMKYLEESVWLHLLIILEDGIIEFYLDGNAMPRGIKSLKGEAITDGPGILRIGAGINGNDRFTGLMQDVRSYERKLTLEEIYELHAMPAKSDLHPISGYLEFRQGETNKSFIISARDDNDEEGEELFILKLVSVYGGARISEENTTARLTIQKSDNANGLFGFTGACIPEIAEEGSTISCVVERTRGALDYVHVFYTISQIETDGINYLVDDFANASGTITFLPWQRSEVLNIYVLDDDIPELNEYFRVTLVSAIPGDGKLGSTPTSGASIDPEKETTDITIKASDHPYGLLQFSTGLPPQPKDAMTLPASSVPHITVEEEDGEIRLLVIRAQGLLGRVTAEFRTVSLTAFSPEDYQNVAGTLEFQPGERYKYIFINITDNSIPELEKSFKVELLNLEGGAELFRVDGSGSGDGDMEFFLPTIHKRASLGVASQILVTIAASDHAHGVFEFSPESLFVSGTEPEDGYSTVTLNVIRHHGTLSPVTLHWNIDSDPDGDLAFTSGNITFEIGQMSANITVEILSDEDPELDKAFSVSILSVSSGSLGAHINATLTVLASDDPYGIFIFSEKNRPVKVEEATQNITLSIIRLKGLMGKVLVSYATLDDMEKPPYFPPNLARATQGRDYIPASGFALFGANQSEATIAISILDDDEPERSESVFIELLNSTLVAKVQSRSIPNSPRLGPKVETIAQLIIIANDDAFGTLQLSAPIVRVAENHVGPIINVTRTGGAFADVSVKFKAVPITAIAGEDYSIASSDVVLLEGETSKAVPIYVINDIYPELEESFLVQLMNETTGGARLGALTEAVIIIEASDDPYGLFGFQITKLIVEEPEFNSVKINLPIIRNSGTLGNVTVQWVATINGQLATGDLRVVSGNVTFAPGETIQTLLLEVLADDVPEIEEVIQVQLTDASGGGTIGLDRIANIIIPANDDPYGTVAFAQMVYRVQEPLERSSCANITVRRSGGHFGRLLLFYSTSDIDVVALAMEEGQDLLSYYESPIQGVPDPLWRTWMNVSAVGEPLYTCATLCLKEQACSAFSFFSASEGPQRFWMTSWISPAVNNSDFWTYRKNMTRVASLFSGQAVAGSDYEPVTRQWAIMQEGDEFANLTVSILPDDFPEMDESFLISLLEVHLMNISASLKNQPTIGQPNISTVVIALNGDAFGVFVIYSISPNTSEDGLFVEVQEQPQTLVELMIHRTGGSLGQVAVEWRVVGGTATEGLDFIGAGEILTFAEGETKKTVILTILDDSEPEDDESIIVSLVYTEGGSRILPSSDTVRVNILANDNVAGIVSFQTASRSVIGHEGEILQFHVIRTFPGRGNVTVNWKIIGQNLELNFANFSGQLFFPEGSLNTTLFVHLLDDNIPEEKEVYQVILYDVRTQGVPPAGIALLDAQGYAAVLTVEASDEPHGVLNFALSSRFVLLQEANITIQLFINREFGSLGAINVTYTTVPGMLSLKNQTVGNLAEPEVDFVPIIGFLILEEGETAAAINITILEDDVPELEEYFLVNLTYVGLTMAASTSFPPRLDSEGLTAQVIIDANDGARGVIEWQQSRFEVNETHGSLTLVAQRSREPLGHVSLFVYAQNLEAQVGLDYIFTPMILHFADGERYKNVNIMILDDDIPEGDEKFQLILTNPSPGLELGKNTIALIIVLANDDGPGVLSFNNSEHFFLREPTALYVQESVAVLYIVREPAQGLFGTVTVQFIVTEVNSSNESKDLTPSKGYIVLEEGVRFKALQISAILDTEPEMDEYFVCTLFNPTGGARLGVHVQTLITVLQNQAPLGLFSISAVENRATSIDIEEANRTVYLNVSRTNGIDLAVSVQWETVSETAFGMGGMDVVFSVFQSFLDESASGWCFFTLENLIYGIMLRKSSVTVYRWQGIFIPVEDLNIENPKTCEAFNIGFSPYFVITHEERNEEKPSLNSVFTFTSGFKLFLVQTIIILESSQVRYFTSDSQDYLIIASQRDDSELTQVFRWNGGSFVLHQKLPVRGVLTVALFNKGGSVFLAISQANARLNSLLFRWSGSGFINFQEVPVSGTTEVEALSSANDIYLIFAKNVFLGDQNSIDIFIWEMGQSSFRYFQSVDFAAVNRIHSFTPASGIAHILLIGQDMSALYCWNSERNQFSFVLEVPSAYDVASVTVKSLNSSKNLIALVGAHSHIYELAYISSHSDFIPSSGELIFEPGEREATIAVNILDDTVPEKEESFKVQLKNPKGGAEIGINDSVTITILSNDDAYGIVAFAQNSLYKQVEEMEQDSLVTLNVERLKGTYGRITIAWEADGSISDIFPTSGVILFTEGQVLSTITLTILADNIPELSEVVIVTLTRITTEGVEDSYKGATIDQDRSKSVITTLPNDSPFGLVGWRAASVFIRVAEPKENTTTLQLQIARDKGLLGDIAIHLRAQPNFLLHVDNQATENEDYVLQETIIIMKENIKEAHAEVSILPDDLPELEEGFIVTITEVNLVNSDFSTGQPSVRRPGMEIAEIMIEENDDPRGIFMFHVTRGAGEVITAYEVPPPLNVLQVPVVRLAGSFGAVNVYWKASPDSAGLEDFKPSHGILEFADKQVTAMIEITIIDDAEFELTETFNTSLISVAGGGRLGDDVVVTVVIPQNDSPFGVFGFEEKTVMIDESLSSDDPDSYVTLTVVRSPGGKGTVRLEWTIDEKAKHNLSPLNGTLHFDETESQKTIVLHTLQDTVLEEDRRFTIQLISIDEVEISPVKGSASIIIRGDKRASGEVGIAPSSRHILIGEPSAKYNGTAIISLVRGPGILGEVTVFWRIFPPSVGEFAETSGKLTMRDEQSAVIVVIQALNDDIPEEKSFYEFQLTAVSEGGVLSESSSTANITVVASDSPYGRFAFSHEQLRVSEAQRVNITIIRSSGDFGHVRLWYKTMSGTAEAGLDFVPAAGELLFEAGEMRKSLHVEILDDDYPEGPEEFSLTITKVELQGRGYDFTIQENGLQIDQPPEIGNISIVRIIIMKNDNAEGIIEFDPKYTAFEVEEDVGLIMIPVVRLHGTYGYVTADFISQSSSASPGGVDYILHGSTVTFQHGQNLSFINISIIDDNESEFEEPIEILLTGATGGAVLGRHLVSRIIIAKSDSPFGVIRFLNQSKISIANPNSTMILSLVLERTGGLLGEIQVNWETVGPNSQEALLPQNRDIADPVSGLFYFGEGEGGVRTIILTIYPHEEIEVEETFIIKLHLVKGEAKLDSRAKDVTLTIQEFGDPNGVVQFAPETLSKKTYSEPLALEGPLLITFFVRRVKGTFGEIMVYWELSSEFDITEDFLSTSGFFTIADGESEASFDVHLLPDEVPEIEEDYVIQLVSVEGGAELDLEKSITWFSVYANDDPHGVFALYSDRQSILIGQNLIRSIQINITRLAGTFGDVAVGLRISSDHKEQPIVTENAERQLVVKDGATYKVDVVPIKNQVFLSLGSNFTLQLVTVMLVGGRFYGMPTILQEAKSAVLPVSEKAANSQVGFESTAFQLMNITAGTSHVMISRRGTYGALSVAWTTGYAPGLEIPEFIVVGNMTPTLGSLSFSHGEQRKGVFLWTFPSPGWPEAFVLHLSGVQSSAPGGAQLRSGFIVAEIEPMGVFQFSTSSRNIIVSEDTQMIRLHVQRLFGFHSDLIKVSYQTTAGSAKPLEDFEPVQNGELFFQKFQTEVDFEITIINDQLSEIEEFFYINLTSVEIRGLQKFDVNWSPRLNLDFSVAVITILDNDDLAGMDISFPETTVAVAVDTTLIPVETESTTYLSTSKTTTILQPTNVVAIVTEATGVSAIPEKLVTLHGTPAVSEKPDVATVTANVSIHGTFSLGPSIVYIEEEMKNGTFNTAEVLIRRTGGFTGNVSITVKTFGERCAQMEPNALPFRGIYGISNLTWAVEEEDFEEQTLTLIFLDGERERKVSVQILDDDEPEGQEFFYVFLTNPQGGAQIVEGKDDTGFAAFAMVIITGSDLHNGIIGFSEESQSGLELREGAVMRRLHLIVTRQPNRAFEDVKVFWRVTLNKTVVVLQKDGVNLMEELQSVSGTTTCTMGQTKCFISIELKPEKVPQVEVYFFVELYEATAGAAINNSARFAQIKILESDESQSLVYFSVGSRLAVAHKKATLISLQVARDSGTGLMMSVNFSTQELRSAETIGRTIISPAISGKDFVITEGTLVFEPGQRSTVLDVILTPETGSLNSFPKRFQIVLFDPKGGARIDKVYGTANITLVSDADSQAIWGLADQLHQPVNDDILNRVLHTISMKVATENTDEQLSAMMHLIEKITTEGKIQAFSVASRTLFYEILCSLINPKRKDTRGFSHFAEVTENFAFSLLTNVTCGSPGEKSKTILDSCPYLSILALHWYPQQINGHKFEGKEGDYIRIPERLLDVQDAEIMAGKSTCKLVQFTEYSSQQWFISGNNLPTLKNKVLSLSVKGQSSQLLTNDNEVLYRIYAAEPRIIPQTSLCLLWNQAAASWLSDSQFCKVVEETADYVECACSHMSVYAVYARTDNLSSYNEAFFTSGFICISGLCLAVLSHIFCARYSMFAAKLLTHMMAASLGTQILFLASAYASPQLAEESCSAMAAVTHYLYLCQFSWMLIQSVNFWYVLVMNDEHTERRYLLFFLLSWGLPAFVVILLIVILKGIYHQSMSQIYGLIHGDLCFIPNVYAALFTAALVPLTCLVVVFVVFIHAYQVKPQWKAYDDVFRGRTNAAEIPLILYLFALISVTWLWGGLHMAYRHFWMLVLFVIFNSLQGLYVFMVYFILHNQMCCPMKASYTVEMNGHPGPSTAFFTPGSGMPPAGGEISKSTQNLIGAMEEVPPDWERASFQQGSQASPDLKPSPQNGATFPSSGGYGQGSLIADEESQEFDDLIFALKTGAGLSVSDNESGQGSQEGGTLTDSQIVELRRIPIADTHL

(19) United States
(12) Patent Application Publication
(10) Pub. No.: US 2003/0003538 A1
(43) Pub. Date: Jan. 2, 2003

(54) Neuropathic pain genes; compositions thereof; related reagents

(76) Inventors: Paul Shartzer Dietrich, Palo Alto, CA (US); Chiao-Chain Huang, Los Altos, CA (US); Carl D. Johnson, Burlingame, CA (US); Lakshmi Sangameswaran, San Jose, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/147,026

(22) Filed: May 15, 2002

Related U.S. Application Data
(62) Division of application No. 09/666,067, filed on Sep. 19, 2000.
(60) Provisional application No. 60/155,702, filed on Sep. 23, 1999.
(60) Provisional application No. 60/198,931, filed on Apr. 21, 2000.

(51) Int. Cl. A01K 67/00; C07H 21/04; C12N 9/16; C12N 5/08; C12P 21/02

(52) U.S. Cl. 435/69.1; 435/320.1; 435/325; 435/196; 435/366; 800/8; 536/23.2

(57) ABSTRACT

The invention provides NPGs from rat and human, reagents related thereto including polynucleotides encoding NPGs, purified polypeptides, and specific antibodies. Methods of making and using these reagents, in particular, methods for screening compounds which modulate NPGs activity are provided. Also provided are methods of diagnosis, kits, and transgenic animals.

### RELATED APPLICATIONS

This U.S. Patent Application claims benefit, under 35 U.S.C. 119(e), to U.S. Provisional Application Serial Nos. 60/155,702, filed Sep. 23, 1999, and 60/198,931, filed Apr. 4, 2000, both of which are incorporated herein by reference in their entirety.

### FIELD OF THE INVENTION

The present invention relates to nucleic acid and amino acid sequences associated with neuorpathic pain and the use of these sequences in diagnosis of various disease states associated with neuropathic pain and cognition and for use as targets for screening compounds useful in the treatment of these disease states.

### BACKGROUND OF THE INVENTION

Pain is the most common reason that patients visit a physician, and such complaints account for millions of visits annually. Nearly 64 million people suffer from trauma-related pain each year. Chronic pain is responsible for billions of dollars in lost workdays. As much as $65 million a year is lost as a result of diminished work productivity. (Bonica, et al. (eds.) (1980) Pain, Discomfort and Humanitarian Care, Elsevier, NY.) Several physiologic types of pain have been defined: somatic, visceral, neuropathic, and complex regional pain syndrome. (Doyle, et al. (eds.) (1997) Textbook of Palliative Medicine, 2^{nd} ed., Oxford University Press, Oxford, England; and Stanton-Hicks, et al. (1995) Pain 63:127.)

Neuropathic pain can be described as pain associated with damage or permanent alteration of the peripheral or central nervous system. Clinical manifestations of neuropathic pain include a sensation of burning or electric shock, feelings of bodily distortion, allodynia and hyperpathia. Peripheral nervous system (PNS) associated neuropathic pain can be divided into two categories: pain affecting single nerves (mononeuropathies); and pain involving the PNS diffusely (polyneuropathies).

Several molecular mechanisms have been implicated in PNS associated neuropathic pain, including activation of certain receptors and ion channels. (Besson (1999) Lancet 353:11610-1615.) With respect to receptors, several G protein coupled receptors (GPCRs) associated with neuropathic pain have been isolated. (Halazy (1999) Exp. Opin. Ther. Patents 9:431-446.)

Similarly, several ion channels associated with nociception, the sensation of pain, have been isolated including, the vanilloid receptor (VR1); ATP-gated ion channels such as the P2X receptors; acid-sensing ion channels such as the ASIC receptors; and sodium channels such as the SNS/PN channels. (See, e.g., McCleskey and Gold (1999) Annu. Rev. Physiol. 61:835-856.)

GPCRs posses unique structure and activities, including seven hydrophobic domains which span the plasma membrane and form a bundle of antiparallel α-helices. Stimulation of these receptors by agonists activates the receptor and allows it to interact with an intracellular G-protein complex. The G-protein complex activates a variety of second messenger molecules which regulate signaling pathways and modulate cellular responses (Lee, M. J. et al (1996) J. Biol. Chem. 271:11272-11279).

Other molecules involved in the physiology of neuropathic pain have yet to be elucidated. The discovery of genes differentially expressed in an animal model of neuropathic pain, presents the opportunity to investigate the mechanisms of this and other neuropathic disorders. Discovery of these molecules satisfies a need in the art by providing new compositions and methods useful in the diagnosis of pain and neurological disease states, and screening of compounds useful in treatment of these disease states.

### DETAILED DESCRIPTION OF THE FIGURE

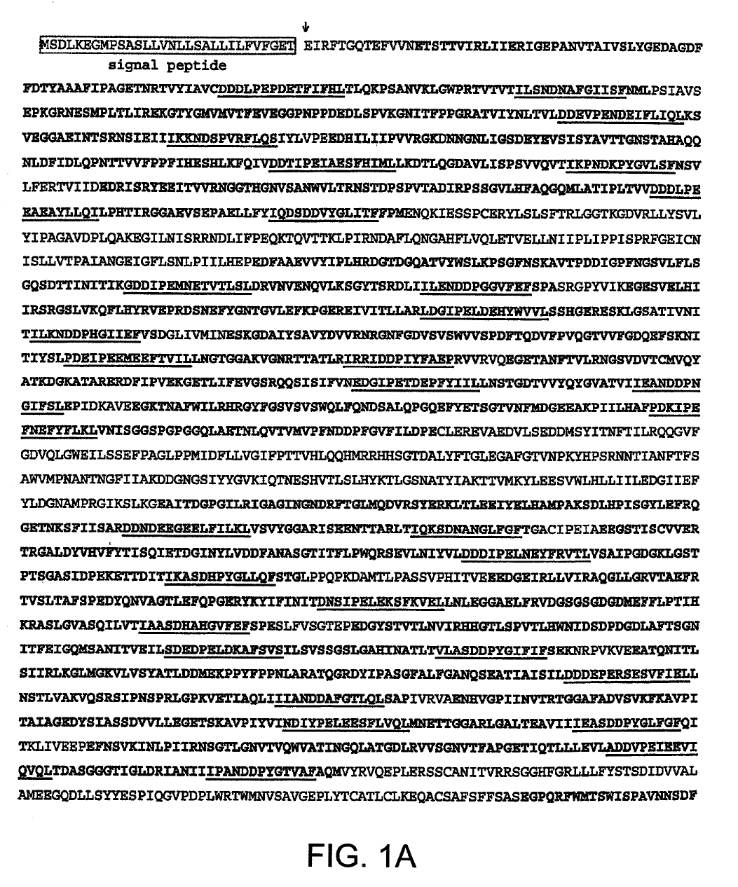

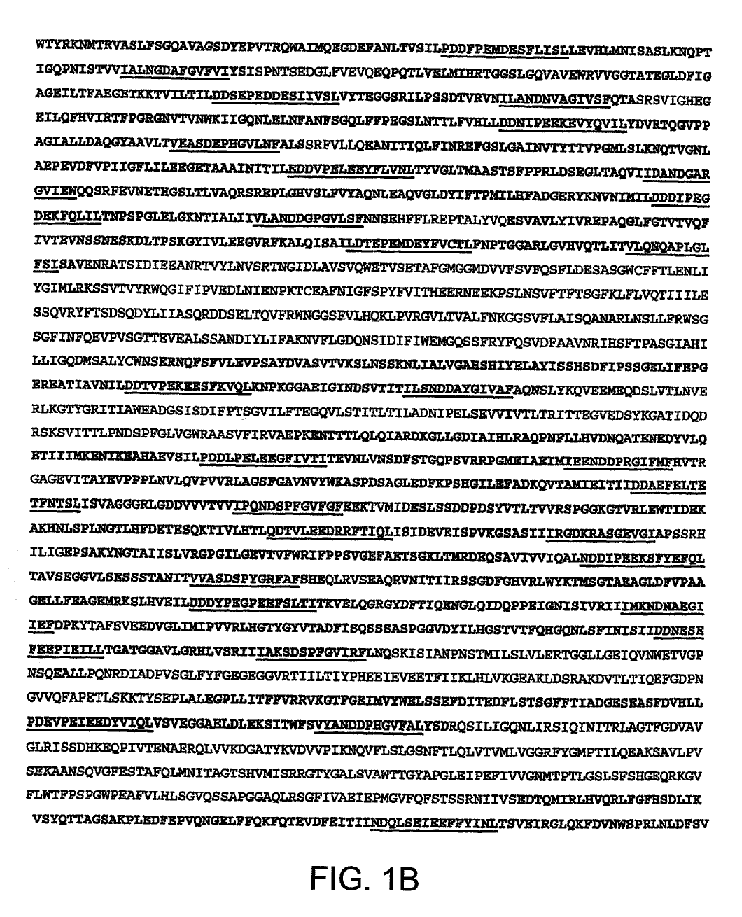

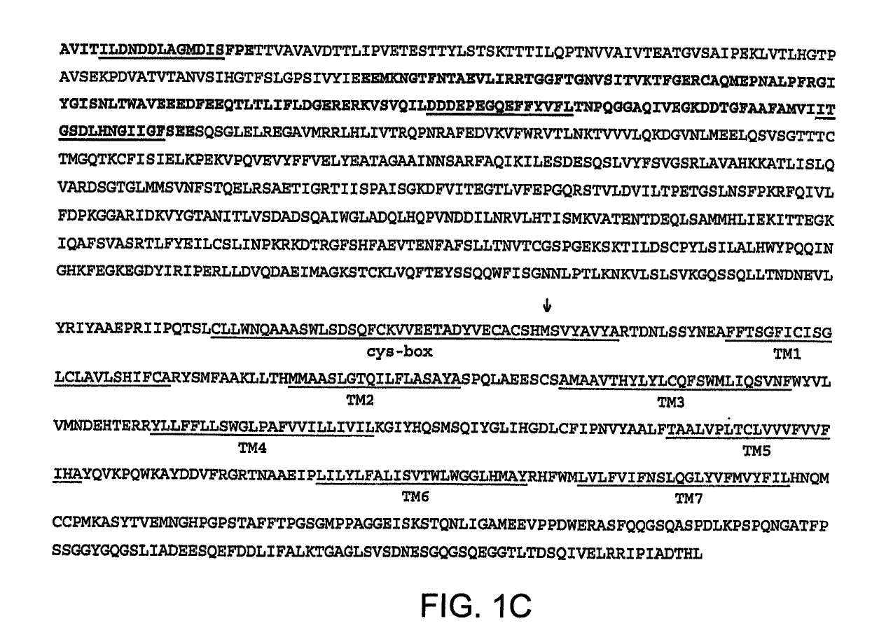

FIGS. 1A-1C show the deduced amino acid sequence for NPG-8 (SEQ ID NO:16). The potential signal peptide is within the dotted line box. The cleavage site is marked by a vertical arrow and a gap. The Na/Ca exchanger Ca⁺⁺ binding domain repeats are in bold with the two acidic amino acid rich segments of each domain underlined. The cystein-rich box just N-terminal of the 7TM region is underlined and the potential cleavage site marked with an arrow. The individual transmembrane domains are underlined.

### SUMMARY OF THE INVENTION

The invention is based on the discovery of new neuropathic pain genes (NPG1, NPG2, NPG3, NPG4, NPG5, NPG6, NPG7, and NPG8, hereinafter NPG) the polynucleotides encoding NPG1-8, and the use of these compositions in screening for compounds effective in treating disease states associated with the nervous system, in particular, neuropathic pain, perphipheral neuropathies, post-traumatic pain, post-surgical pain, pain associated with cancer, pain associated with chemotheraphy; and neurological disease states including, but not limited to, cognitive disease states, such as Alzheimer's disease and dementia, and the use of these compositions for diagnosis of these disease states. In particular, the present invention provides expression vectors, host cells, antibodies, diagnostic kits, and transgenic/knockout animals.

The invention features an isolated polynucleotide encoding a neuropathic pain gene (NPG) polypeptide. The invention further provides an isolated polynucleotide, encoding an NPG polypeptide wherein the polynucleotide encodes an NPG polypeptide comprising the amino acid sequence of SEQ ID NO:2, 4, 6, 8, 10, 12, 14 or 16. In certain embodiments, the polynucleotide is detectably labeled or is complementary to the polynucleotide encoding an NPG polypeptide. The complementary polynucleotide can also be detectably labeled. In another embodiment, the polynucleotide comprises the nucleic acid sequence of SEQ ID NO:1, 3, 5, 7, 9, 11, 13, or 15.

The present invention encompasses an expression vector comprising the polynucleotide encoding an NPG polypeptide. Also contemplated is a host cell comprising the polynucleotide encoding an NPG polypeptide. The host cell can be a prokaryotic or eukaryotic cell. The invention further comprises a method of producing an NPG polypeptide comprising the steps of: culturing the host cell comprising the expression vector comprising the polynucleotide encoding an NPG polypeptide under conditions suitable for expression of the NPG polypeptide; and recovering the polypeptide from the host cell.

The present invention also contemplates a method of detecting a polynucleotide encoding an NPG polypeptide in a sample containing nucleic acid material, comprising: contacting the sample with a polynucleotide which is the complement of the polynucleotide encoding an NPG polypeptide, wherein the complement is detectably labeled, under conditions suitable for formation of a hybridization complex; and detecting the complex, wherein the presence of the complex is indicative of the presence of the polynucleotide encoding the polypeptide in the sample.

The present invention provides a diagnostic test kit comprising: the polynucleotide comprising SEQ ID NO:1, 3, 5, 7, 9, 11, 13, or 15; and instructions for conducting the diagnostic test.

The present invention encompasses a method of screening for a compound that modulates NPG activity comprising contacting an NPG, or fragment thereof with the compound and detecting modulation of NPG activity. In certain embodiments, the NPG is expressed on a cell or tissue or immobilized on a solid support. The compound can be an antagonist of NPG activity or an agonist of activity.

The present invention provides an isolated NPG polypeptide comprising the amino acid sequence of SEQ ID NO:2, 4, 6, 8, 10, 12, 14, or 16. The polypeptide is recombinantly produced or synthetically produced. The present invention also provides an isolated antibody which specifically binds to the polypeptide of SEQ ID NO:2, 4, 6, 8, 10, 12, 14, or 16.

The present invention encompasses a transgenic nonhuman mammal comprising the polynucleotide encoding an NPG polypeptide. The transgenic nonhuman mammal can also comprise the polynucleotide which is the complement of the polynucleotide encoding NPG which is capable of hybridizing to a polynucleotide encoding NPG, thereby reducing expression of NPG.

### DETAILED DESCRIPTION OF THE INVENTION

Before the present proteins, nucleotide sequences, and methods are described, it is to be understood that the present invention is not limited to the particular methodologies, protocols, cell lines, vectors, and reagents described, as these may vary. It is also understood that the terminology used herein is for the purpose of describing particular embodiments only, and is not to limit the scope of the present invention.

The singular forms “a,” “an,” and “the” include plural reference unless the context clearly dictates otherwise.

All technical and scientific terms used herein have the same meanings as commonly understood by one of ordinary skill in the art to which this invention pertains. The practice of the present invention will employ, unless otherwise indicated, conventional techniques of protein chemistry and biochemistry, molecular biology, microbiology and recombinant DNA technology, which are within the skill of the art. Such techniques are explained fully in the literature.

Although any machines, materials, and methods similar or equivalent to those described herein can be used to practice or test the present invention, the preferred machines, materials, and methods are now described. All patents, patent applications, and publications mentioned herein, whether supra or infra, are each incorporated by reference in its entirety.

Definitions

“NPG” refers to the amino acid sequences of substantially purified NPG obtained from any species particularly mammalian species, including bovine, ovine, porcine, murine, equine, and preferably the human species, from any source, whether natural, synthetic, semi-synthetic, or recombinant.

“Agonist” refers to a molecule which, when bound to NPG, or is within proximity of NPG, modulates the activity of NPG by increasing or prolonging the duration of the effect of NPG. Agonists can include proteins, nucleic acids, carbohydrates, organic compounds, inorganic compounds, or any other molecules which modulate the effect of NPG.

An “allelic variant” is an alternative form of the gene encoding NPG. Allelic variants may result from at least one mutation in the nucleic acid sequence and may result in altered mRNAs or in a polypeptide whose structure or function may or may not be altered. Any given recombinant gene may have none, one, or many allelic forms. Common mutational changes which give rise to allelic variants are generally ascribed to natural deletions, additions, or substitutions of nucleotides. Each of these types of changes may occur alone, or in combination with the others, one or more times in a given sequence.

“Amplification” relates to the production of additional copies of a nucleic acid sequence. Amplification can be carried out using polymerase chain reaction (PCR) technologies or other methods well known in the art.

“Antagonist” refers to a molecule which, when bound to NPG or within close proximity, decreases the amount or the duration of the biological or immunological activity of NPG. Antagonists may include proteins, nucleic acids, carbohydrates, antibodies, organic compounds, inorganic compounds, or any other molecules which exert an effect on NPG activity.

“Antibody” can be an intact molecule or fragments thereof, such as Fab, F(ab)₂, and Fv fragments, which are capable of binding an epitopic determinant. The antibody can be polyclonal, monoclonal, or recombinantly produced.

The terms “antigenic determinant” or “epitopic determinant” refer to the fragment of a molecule that makes contact with a particular antibody.

The term “antisense” refers to any composition containing nucleic acids which is complementary to the “sense” strand of a specific nucleic acid molecule. Antisense molecules may be produced by any method including synthesis or transcription. Once introduced into a cell, the complementary nucleotides combine with natural sequences produced by the cell to form duplexes and to block either transcription or translation. The designation “negative” can refer to the antisense strand, and the designation “positive” can refer to the sense strand.

A “coding sequence” is a polynucleotide sequence that is transcribed into mRNA and translated into a polypeptide. The boundaries of the coding sequence are determined by a translation start codon at the 5′-terminus and a translation stop codon at the 3′-terminus. A coding sequence can include, but is not limited to, mRNA, cDNA, synthetic DNA, and recombinant polynucleotide sequences. Also included is genomic DNA where the coding sequence is interrupted by introns.

“Complementary” and “complementarity” refer to the natural binding of polynucleotides to other polynucleotides by base pairing. For example, the sequence “5′ A-C-G-T 3′” will bind to the complementary sequence “3′ T-G-C-A 5′.”

Complementarity between two single stranded molecules may be “partial,” such that only some of the nucleic acids bind, or it may be “complete,” such that total complementarity exists between the single stranded molecules.

A “composition comprising a given polynucleotide sequence” and a “composition comprising a given amino acid sequence” refer broadly to any composition containing the given polynucleotide or amino acid sequence.

The term “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

The phrase “correlates with expression of a polynucleotide” indicates that the detection of the presence of nucleic acids, the same or related to a nucleic acid sequence encoding NPG, e.g., by northern analysis, dot blot, or RT-PCR, is indicative of the presence of nucleic acids encoding NPG in a sample, and thereby correlates with expression of the transcript from the polynucleotide encoding NPG.

The phrase “detectably labeled” as used herein means joining, either covalently or non-covalently to the polynucleotides, polypeptides, or antibodies of the present invention, a substance which provides for a detectable signal. A wide variety of labels and conjugation techniques are well known in the art. Suitable labels include radionuclides, e.g., ³²P, ³⁵S, ³H, enzymes, substrates, cofactors, inhibitors, fluorescent moieties, chemiluminescent moieties, magnetic particles, and the like.

The phrase “disease state” means any disease, condition, disorder, symptom, or indication.

The term “expression” as used herein intends both transcriptional and translational processes, i.e., the production of messenger RNA and/or the production of protein therefrom.

The term “hybridization complex” refers to a complex formed between two nucleic acid sequences by virtue of the formation of hydrogen bonds between complementary bases. A hybridization complex may be formed in solution (conditions calculated by performing, e.g., Cot or Rot) or formed between one nucleic acid sequence present in solution and another nucleic acid sequence immobilized on a solid support (e.g., paper, membranes, filters, chips, pins, glass slides, or any other appropriate substrate to which cells or their nucleic acids have been fixed.)

An “isolated polynucleotide” that encodes a particular polypeptide refers to a polynucleotide that is substantially free of other nucleic acid molecules that do not encode the subject polypeptide; however, the molecule may include functionally and/or structurally conservative mutations as defined herein.

The term “modulate” refers to a change in the activity of NPG. For example, modulation may cause an increase or a decrease in protein activity, binding characteristics, or any other biological, functional, or immunological properties of NPG. The ability to modulate the activity of NPG can be exploited in assays to screen for organic, inorganic, or biological compounds which affect the above properties of NPG.

The term “neuropathy” refers to a functional disturbance or pathological change in the peripheral nervous system. Known etiologies include complications of other diseases, e.g., diabetes, cancer, etc., or of toxic states, e.g., arsenic, isoniazid, lead, nitrofurantoin, etc. poisoning.

“Nucleic acid” and “nucleic acid sequence” refer to a nucleotide, oligonucleotide, polynucleotide, or any fragment thereof. These phrases also refer to DNA or RNA of genomic or synthetic origin which may be single stranded or double stranded and may represent the sense of the antisense strand, a peptide nucleic acid (PNA), or any DNA-like or RNA-like material. In this context, “fragments” refer to those nucleic acids which, when translated, would produce polypeptides retaining some functional characteristic, e.g., antigenicity, or structural domain, e.g., ion channel domain, characteristic of the full-length polypeptide.

The terms “operably associated” and “operably linked” refer to functionally related but heterologous nucleic acid sequences. A promoter is operably associated or operably linked with a coding sequence if the promoter controls the translation or expression of the encoded polypeptide. While operably associated or operably linked nucleic acid sequences can be contiguous and in the same reading frame, certain genetic elements, e.g., repressor genes, are not contiguously linked to the sequence encoding the polypeptide but still bind to operator sequences that control expression of the polypeptide.

An “oligonucleotide” refers to a nucleic acid molecule of at least about 6 to nucleotides, preferably about 15 to 30 nucleotides, and most preferably 20 to 25 nucleotides, which can be used in PCR amplification or in a hybridization assay. “Oligonucleotide” is substantially equivalent to the terms “amplimer,” “primer,” “oligomer,” and “probe” as these terms are commonly defined in the art.

“Peptide nucleic acid” (PNA) refers to an antisense molecule or anti-gene agent which comprises an oligonucleotide of at least 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

The phrases “percent identity” and “% identity” refer to the percentage of sequence similarity found by a comparison or alignment of two or more amino acid or nucleic acid sequences. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman (1981) Advances in Appl. Math. 2:482-489, for peptide analysis. Programs for determining nucleotide sequence identity are available in the Wisconsin Sequence Analysis Package, Version 8 (Genetics Computer Group, Madison, Wis.) for example, the BLAST, BESTFIT, FASTA, and GAP programs, which also rely on the Smith and Waterman algorithm. These programs are readily utilized with the default parameters recommended by the manufacturer and described in the Wisconsin Sequence Analysis Package referred to above. Other programs for calculating identity or similarity between sequences are known in the art.

“Recombinant host cells,” “host cells,” “cells,” “cell lines,” “cell cultures,” and other such terms denoting cell lines cultured as unicellular entities refer to cells which can be, or have been, used as recipients for recombinant vectors or other transfer DNA, immaterial of the method by which the DNA is introduced into the cell or the subsequent disposition of the cell. The terms include the progeny of the original cell which has been transfected. Cells in primary culture as well as cells such as oocytes also can be used as recipients.

A “reporter gene” is a gene that, upon expression, confers a phenotype on a cell expressing the reporter gene, such that the cell can be identified under appropriate conditions. For example, the reporter gene may produce a polypeptide product that can be easily detected or measured in a routine assay. Suitable reporter genes known in the art which confer this characteristic include those that encode chloramphenicol acetyl transferase (CAT activity), β-galactosidase, luciferase, alkaline phosphatase, human growth hormone, fluorescent proteins, such as green fluorescent protein (GFP), and others. Indeed, any gene that encodes a protein or enzyme that can readily be measured, for example, by an immunoassay such as an enzyme-linked immunosorbent assay (ELISA) or by the enzymatic conversion of a substrate into a detectable product, and that is substantially not expressed in the host cells (specific expression with no background) can be used as a reporter gene to test for promoter activity. Other reporter genes for use herein include genes that allow selection of cells based on their ability to thrive in the presence or absence of a chemical or other agent that inhibits an essential cell function. Suitable markers, therefore, include genes coding for proteins which confer drug resistance or sensitivity thereto, or change the antigenic characteristics of those cells expressing the reporter gene when the cells are grown in an appropriate selective medium. For example, reporter genes include: cytotoxic and drug resistance markers, whereby cells are selected by their ability to grow on media containing one or more of the cytotoxins or drugs; auxotrophic markers by which cells are selected by their ability to grow on defined media with or without particular nutrients or supplements; and metabolic markers by which cells are selected for, e.g., their ability to grow on defined media containing the appropriate sugar as the sole carbon source. These and other reporter genes are well known in the art.

A “change in the level of reporter gene product” is shown by comparing expression levels of the reporter gene product in a cell exposed to a candidate compound relative to the levels of reporter gene product expressed in a cell that is not exposed to the test compound and/or to a cell that is exposed to a control compound. The change in level can be determined quantitatively for example, by measurement using a spectrophotometer, spectrofluorometer, luminometer, and the like, and will generally represent a statistically significant increase or decrease in the level from background. However, such a change may also be noted without quantitative measurement simply by, e.g., visualization, such as when the reporter gene is one that confers the ability on cells to form colored colonies on chromogenic substrates.

The term “sample” is used in its broadest sense. A sample suspected of containing nucleic acids encoding NPG, or fragments thereof, or NPG polypeptide may comprise a bodily fluid; an extract from a cell chromosome, organelle, or membrane isolated from a cell; an intact cell; genomic DNA, RNA, or cDNA, in solution or bound to a substrate; a tissue; a tissue print; etc.

“Stringent conditions” refers to conditions which permit hybridization between polynucleotides and the claimed polynucleotides. Stringent conditions can be defined by salt concentration, the concentration of organic solvent, e.g., formamide, temperature, and other conditions well known in the art.

“Subject” means mammals and non-mammals. Mammals means any member of the Mammalia class including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

The term “substantially purified,” when referring to a polypeptide, indicates that the polypeptide is present in the substantial absence of other similar biological macromolecules.

The term “transfection” refers to the insertion of an exogenous polynucleotide into a host cell, irrespective of the method used for the insertion, or the molecular form of the polynucleotide that is inserted. The insertion of a polynucleotide per se and the insertion of a plasmid or vector comprised of the exogenous polynucleotide are included. The exogenous polynucleotide may be directly transcribed and translated by the cell, maintained as a nonintegrated vector, for example, a plasmid, or alternatively, may be stably integrated into the host genome.

The term “transformed” refers to any known method for the insertion of foreign DNA or RNA sequences into a host prokaryotic cell. The term “transfected” refers to any known method for the insertion of foreign DNA or RNA sequences into a host eukaryotic cell. Such transformed or transfected cells include stably transformed or transfected cells in which the inserted DNA is rendered capable of replication in the host cell. They also include transiently expressing cells which express the inserted DNA or RNA for limited periods of time. The transformation or transfection procedure depends on the host cell being transformed. It can include packaging the polynucleotide in a virus as well as direct uptake of the polynucleotide, such as, for example, lipofection or microinjection. Transformation and transfection can result in incorporation of the inserted DNA into the genome of the host cell or the maintenance of the inserted DNA within the host cell in plasmid form. Methods of transformation are well known in the art and include, but are not limited to, viral infection, electroporation, lipofection, and calcium phosphate mediated direct uptake.

“Treating” or “treatment” of a disease state includes: 1) preventing the disease state, i.e. causing the clinical symptoms of the disease state not to develop in a subject that may be exposed to or predisposed to the disease state, but does not yet experience or display symptoms of the disease state; 2) inhibiting the disease state, i.e., arresting the development of the disease state or its clinical symptoms; 3) or relieving the disease state, i.e., causing temporary or permanent regression of the disease state or its clinical symptoms.

A “variant” of NPG polypeptides refers to an amino acid sequence that is altered by one or more amino acid residues. The variant may have “conservative” changes, wherein a substituted amino acid has similar structural or chemical properties (e.g., replacement of leucine with isoleucine.) More rarely, a variant may have “nonconservative” changes (e.g., replacement of glycine with tryptophan.) Analogous minor variations may also include amino acid deletion or insertions, or both. Guidance in determining which amino acid variations may be substituted, inserted, or deleted without abolishing biological function may be found using programs well known in the art, for example, LASERGENE software (DNASTAR).

The term “variant” when used in the context of a polynucleotide sequence, may encompass a polynucleotide sequence related to NPG. This definition may include, for example “allelic” (as defined above), “splice,” “species,” “polymorphic,” or “degenerate” variants. A splice variant may have significant identity to a reference molecule, but will generally have a greater of less number polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity to each other. A polymorphic variant is a variation in the polynucleotide sequence of a particular gene between individuals within a given species. Polymorphic variants may also encompass “single nucleotide polymorphisms” (SNPs) in which the polynucleotide sequence varies by one base. The presence of SNPs may be indicative of, for example, a certain population, a disease state, or a propensity for a disease state. A degenerate variant encompasses a multitude of polynucleotides which encode NPG polypeptides. The degenerate variants may occur naturally or may be produced synthetically. Synthetic degenerate variants are made in accordance with the standard triplet genetic code as applied to the polynucleotide sequence of naturally occurring NPG, and all such variations are to be considered as being specifically disclosed.

A “vector” is a replicon in which another polynucleotide segment is attached, such as to bring about the replication and/or expression of the attached segment. The term includes expression vectors, cloning vectors, and the like.

The Invention

The present invention is based on the discovery of a new NPG, the polynucleotides encoding NPG, and the use of these compositions for screening compounds useful in the treatment or prevention of disease states associated with neuropathic pain, including, but not limited to, peripheral neuropathies, post-traumatic pain, post-surgical pain, diabetic neuropathy, cancer neuropathy, pain associated with chemotherapy, toxic neuropathy, and the like.

Table 1 describes the clone number, homologue, tissue distribution, domains of particular interest, and the corresponding sequence identifier.

TABLE 1
Neuropathic pain genes

| Molecule | | | | | |
|---|---|---|---|---|---|
| Name | | Homolog | | | |
| (Clone | Type of | (Accession | Percent | Tissue | Sequence |
| number) | Sequence | number) | Identity | Distribution | Identifier |
| NPG-1 | Nucleic | Thioredoxin | 65% | Highest in | SEQ ID |
| (IV-33) | and | Domain | | brain, heart, | NO: 1 |
| | amino | (AL021396) | | liver, and | SEQ ID |
| | acid | | | testis | NO: 2 |
| NPG-2 | Nucleic | LIM domain | 63% | Highest in | SEQ ID |
| (IV-56) | and | (U49957), | | heart, liver, | NO: 3 |
| | amino | ajuba, | 30% | and spleen | SEQ ID |
| | acid | CE15, | | | NO: 4 |
| | | zyxin | | | |
| | | (AF097511) | | | |
| NPG-3 | Nucleic | TPR domain | 28% | Brain | SEQ ID |
| (IV-63) | and | (AF016427) | | | NO: 5 |
| | amino | | | | SEQ ID |
| | acid | | | | NO: 6 |
| NPG-4 | Nucleic | VT4 | 54% | Highest in | SEQ ID |
| (IV-65) | and | (U19346), | | brain, spinal | NO: 7 |
| | amino | yemanuclein | 44% | cord, heart, | SEQ ID |
| | acid | (X63503) | | kidney, | NO: 8 |
| | | | | skeletal | |
| | | | | muscle, and | |
| | | | | testis | |
| NPG-5 | Nucleic | GS2 | 41% | Spinal cord, | SEQ ID |
| (IV-69) | and | (Z97055) | | kidney | NO: 9 |
| | amino | | | | SEQ ID |
| | acid | | | | NO: 10 |
| NPG-6 | Nucleic | KIAA0686 | 81% | Spinal cord | SEQ ID |
| (IV-71) | and | (AB014586), | | | NO: 11 |
| | amino | Na+/Ca++ | 26% | | SEQ ID |
| | acid | Exchanger | | | NO: 12 |
| | | (P70414), | | | |
| | | latrotoxin | 20% | | |
| | | receptor | | | |
| | | (G3766207) | | | |
| NPG-7 | Nucleic | KIAA0871 | 96% | Brain, spinal | SEQ ID |
| (IV-75) | and | protein | | cord | NO: 13 |
| | amino | (AB020678), | | | SEQ ID |
| | acid | rap2 | 36% | | NO: 14 |
| | | Interacting- | | | |
| | | protein 8 | | | |
| | | (AC002457) | | | |
| NPG-8 | Nucleic | Very Large | 100% | Brain, | SEQ ID |
| (human | and | G Protein | over 3′ | pituitary | NO: 15 |
| ortholog | amino | Receptor | 6 kb | | SEQ ID |
| of | acid | (AF055084) | portion | | NO: 16 |
| NPG-6) | | | | | |

The sequence of NPG-6 (SEQ ID Nos:11 and 12) was analyzed by computer algorithms, e.g., PHD (Rost and Burhard (1996) Meth. Enzymol. 266:525-539), BLAST, etc. Computer analysis revealed seven transmembrane domains (TM) characteristic of G protein coupled receptors (GPCR). Further analysis showed NPG-6 to be most closely related to Family B, Group 4 GPCRs, which includes the latrophilin-1 receptor, the EMR1 hormone receptor, and CD97. GenBank Accession number AB014586, corresponding to KIAA0686, shows high identity to a fragment of NPG-6.

The N-terminal region of Family B, Group 4 GPCR are characteristically longer than Family B, Groups 1-3. Additional BLAST analysis of the the N-terminal region of NPG-6 showed identity to the NCX class of Na⁺/Ca⁺⁺ exchangers (e.g., GenBank Accesion No. P70414), which are found in a variety of organisms. One characteristic of this class of ion exchangers is the presence of highly conserved NIbeta domains.

Structurally, NPG-6 contains at least six NIβ domains in the extracellular NH-terminus and 7 transmembrane domains proximal to the COOH-terminus. The NIβ domains encompass residues from: 89 through about 189; about 209 through about 309; about 569 through about 669; about 805 through about 905; and about 1060 through about 1160, each of SEQ ID NO:12. The transmembrane domains encompass residues from: about 1481 through about 1501; about 1513 through about 1533; about 1543 through about 1563; about 1585 through about 1605; about 1634 through about 1654; about 1678 through about 1698; and 1704 through about 1724, each of SEQ ID NO:12. An extracellular fragment runs from about amino acid residue 1 through about residue 1478.

In situ hybridization performed on rat brain sections showed that NPG-6 prominently localized in the ventrocaudal striatum, an area known to have extensive connections with lateral mesocortical regions. NPG-8 also localized in the parafascicular nucleus, a part of the intralaminar nuclear complex of the thalamus, as well as within the ependyma lining of the ventricles.

NPG-8 is the human ortholog of NPG-6. BLAST analysis with the amino acid sequence (SEQ ID NO:16) revealed strong homology to the latrotoxin and related GPCR family B receptors comprising approximately the last 500 amino acids at the carboxy end of the sequence. Seven potential transmembrane domains were identified as well as a conserved cysteine-rich sequence, proximal to the first TM, which is a potential proteolytic processing site in the latrotoxin receptor family. Searches using portions of NPG-8 revealed identity to various publicly available genomic sequences (e.g., GenBank Accession No. AC027323), some of which mapped to human chromosome 5.

The cytoplasmic amino terminal domain of NPG-8 contains several domains with homology to the high affinity calcium-binding domain of the NCX class of Na⁺/Ca²⁺ exchangers. (See, e.g., Levitsky, D. O. et al (1994) J. Biol. Chem. 269:22847-22852; and Dyck, C. et al (1998) J. Biol. Chem. 273:12981-12987.) Within each domain, the strongest homology was to two acidic amino acid rich stretches of 12 to 16 amino acids, separated by approximately 15 to 40 amino acids, that are thought to play a critical role in calcium binding (See FIG. 1). Homology was also found to an aggregation protein from a marine sponge (GenBank Accession No. AF020903), which also contains multiple calcium-binding domain homologous regions. GenBank Accession No. AF055084 (Very Large G-protein Receptor; VLGR) was found to have high sequence identity to a portion of NPG-8, with large differences noted in the 5′ end of VRL.

The invention also encompasses nucleic or amino acid variants of NPG. A preferred variant is one which has at least about 80%, more preferably at least about 90%, and most preferably at least about 95% amino acid or nucleic acid identity to the corresponding NPG sequence, and which contains at least one functional or structural characteristic of NPG.

Polynucleotides

Although nucleotide sequences which encode NPG and its variants are preferably capable of hybridizing to the nucleotide sequence of the naturally occurring NPG under appropriately selected conditions of stringency, it may be advantageous to produce nucleotide sequence encoding NPG or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding NPG and its derivatives without altering the encoded amino acid include the production of RNA transcripts having more desirable properties, such as greater half-life or stability for improved translation, than transcripts produced from the naturally occurring sequence.

Also encompassed by the invention are polynucleotides that are capable of hybridizing to the claimed polynucleotide sequences, and, in particular, to those shown in SEQ ID NO:1, 3, 5, 7, 9, 11, 13, or 15, under various conditions of stringency. (See, e.g., Wahl, G. M. and S. L. Berger (1987) Methods Enzymol. 152:399-401; Kimmel, A. R. (1987) Methods Enzymol. 152:507-511.) For example, stringent salt concentration will ordinarily be less that about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., preferably at least about 37° C., and more preferably 42° C. Varying additional parameters such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a preferred embodiment, hybridization will occur at 37° C. in 500 mM NaCl, 50 mM trisodium citrate, 1% SDS, 35% formamide, and 100 μg/ml denatured salmon sperm DNA (ssDNA). In a more preferred embodiment, hybridization will occur at 42° C. in 250 mM NaCl, 25 mM trisodium citrate, 1% SDS, 50% formamide, and 200 μg/ml denatured ssDNA. Useful variations of these conditions will be readily apparent to those skilled in the art.

The washing steps which follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and more preferably less than about 15 mM NaCl and 1.5 mM trisodium citrate. Stringent temperature conditions for the wash step will ordinarily include temperature of at least about 25° C., preferably of at least about 42° C., and more preferably of at least about 68° C. Generally the wash step will occur at 25° C. in 30 mM NaCl, 3 mM trisodium citrate, and 0.1% SDS. Preferably, the wash step will occur at 42° C. in 15 mM NaCl, 1.5 mM trisodium citrate, and 0.1% SDS. More preferably, the wash step will occur at 68° C., in 15 mM NaCl, 1.5 mM trisodium citrate, and 0.1% SDS. Additional variations on these conditions will be readily apparent to those skilled in the art.

In another embodiment, polynucleotide sequences encoding all or part of NPG may be synthesized using chemical methods well known in the art. (See, e.g., Caruthers, M. H. et al. (1980) Nucl. Acids Res. Symp. Ser. 7:215-223, Horn, T. et al. (1980) Nucl. Acids Res. Symp. Ser. 7:225-232.)

The present invention further covers recombinant polynucleotides and fragments having a DNA sequence identical to or highly homologous to the isolated polynucleotides of NPG. In particular, the sequences will often be operably linked to DNA segments which control transcription, translation, and DNA replication. Alternatively, recombinant clones derived from the genomic sequences, e.g., containing introns, will be useful for transgenic and knock-out studies, including transgenic cells, organisms, and knock-out animals, and for gene therapy. (See, e.g., Goodnow (1992) “Transgenic Animals” in Roitt (ed.) Encyclopedia of Immunology, Academic Press, San Diego, Calif., pp. 1502-1504; Travis (1992) Science 254:707-710; Capecchi (1989) Science 244:1288-1292; Robertson (ed.) (1987) Teratocarcinomas and Embryonic Stem Cells: A Practical Approach, IRL Press, Oxford; Rosenberg (1992) J. Clinical Oncology 10:180-199; Hogan, et al. (eds.) (1994) Manipulating the Mouse Embryo: A Laboratory Manual, 2^{nd} edition, Cold Spring Harbor Press, NY; Wei (1997) Ann. Rev. Pharmacol. Toxicol. 37:119-141; and Rajewsky, et al. (1996) J. Clin. Inves. 98:S51-S53.)

Examples of these techniques include: 1) Insertion of normal or mutant versions of DNA encoding NPG or homologous animal versions of these genes, by microinjection, retroviral infection, or other means well known to those skilled in the art, into appropriate fertilized embryos in order to produce a transgenic animal (see, e.g., Hogan, supra); and 2) homologous recombination (see, e.g., Capecchi, supra; and Zimmer and Gruss (1989) Nature 338:150-153) of mutant or normal, human or animal versions of these genes with the native gene locus in transgenic animals to alter the regulation of expression or the structure of NPG.

The technique of homologous recombination is well known in the art. It replaces the native gene with the inserted gene and is thus useful for producing an animal that cannot express native receptor but does express, for example, an inserted mutant receptor, which has replaced the native receptor in the animal's genome by recombination, resulting in underexpression of the receptor.

Microinjection adds genes to the genome, but does not remove them, and so is useful for producing an animal which expresses its own and added receptors, resulting in overexpression of the receptor. One means available for producing a transgenic animal, with a mouse as an example, is as follows: Female mice are mated, and the resulting fertilized eggs are dissected out of their oviducts. The eggs are stored in an appropriate medium such as M2 medium (see, e.g., Hogan, supra). DNA or cDNA encoding NPG is purified from an appropriate vector by methods well known in the art. Inducible promoters may be fused with the coding region of the DNA to provide an experimental means to regulate expression of the trans-gene. Alternatively, or in addition, tissue specific regulatory elements may be fused with the coding region to permit tissue-specific expression of the trans-gene. The DNA, in an appropriately buffered solution, is put into a microinjection needle (which may be made from capillary tubing using a pipet puller) and the egg to be injected is put in a depression slide. The needle is inserted into the pronucleus of the egg, and the DNA solution is injected. The injected egg is then transferred into the oviduct of a pseudopregnant mouse (a mouse stimulated by the appropriate hormones to maintain pregnancy but which is not actually pregnant), where it proceeds to the uterus, implants, and develops to term. As noted above, microinjection is not the only method for inserting DNA into the egg cell, and is used here only for exemplary purposes.

Since the normal action of receptor-specific drugs is to activate or to inhibit the receptor, the transgenic animal model systems described above are useful for testing the biological activity of drugs directed against NPG even before such drugs become available. These animal model systems are useful for predicting or evaluating possible therapeutic applications of drugs which activate or inhibit NPG by inducing or inhibiting expression of the native or trans-gene and thus increasing or decreasing expression of normal or mutant NPG in the living animal. Thus, a model system is produced in which the biological activity of drugs directed against NPG are evaluated before such drugs become available.

The transgenic animals which over- or under-produce NPG indicate, by their physiological state, whether over- or underproduction of NPG is therapeutically useful. It is therefore useful to evaluate drug action based on the transgenic model system. One use is based on the fact that it is well known in the art that a drug such as an antidepressant acts by blocking neurotransmitter uptake, and thereby increases the amount of neurotransmitter in the synaptic cleft. The physiological result of this action is to stimulate the production of less receptor by the affected cells, leading eventually to underexpression. Therefore, an animal which underexpresses receptor is useful as a test system to investigate whether the actions of such drugs which result in under expression are in fact therapeutic. Another use is that if overexpression is found to lead to abnormalities, then a drug which down-regulates or acts as an antagonist to NPG is indicated as worth developing, and if a promising therapeutic application is uncovered by these animal model systems, activation or inhibition of NPG is achieved therapeutically either by producing agonist or antagonist drugs directed against NPG or by any method which increases or decreases the expression of NPG in man.

Polypeptides

The predicted sequence NPG amino acid sequence is shown in SEQ ID NO:2, 4, 6, 8, 10, 12, 14, or 16. The peptide sequences allow preparation of peptides to generate antibodies to recognize such segments, and various different methods may be used to prepare such peptides. As used herein NPG shall encompass, when used in a protein context, a protein having an amino acid sequence of SEQ ID NO:2, 4, 6, 8, 10, 12, 14, or 16, or a significant fragment of such a protein. It also refers to a vertebrate, e.g., mammal, including human, derived polypeptide which exhibits similar biological function, e.g., antigenic, or interacts with NPG specific binding components, e.g., specific antibodies.

The term polypeptide, as used herein, includes a significant fragment or segment, and encompasses a stretch of amino acid residues of at least about 8 amino acids, generally at least 10 amino acids, more generally at least 12 amino acids, often at least 14 amino acids, more often at least 16 amino acids, typically at least 18 amino acids, more typically at least 20 amino acids, usually at least 22 amino acids, more usually at least 24 amino acids, preferably at least 26 amino acids, more preferably at least 28 amino acids, and, in particularly preferred embodiments, at least about 30 or more amino acids. The segments may have lengths of at least 37, 45, 53, 61, 70, 80, 90, etc., and often will encompass a plurality of such matching sequences. The specific ends of such a segment will be at any combinations within the protein. Preferably the fragment will encompass structural domains, as described above, or unique regions useful in generation of binding compositions with specificity for NPG. In another embodiment of the invention, natural, modified, or recombinant nucleic acid sequences encoding NPG may be ligated to a heterologous sequence to encode a fusion protein. For example, to screen peptide libraries for inhibitors of NPG activity, it may be useful to encode a chimeric NPG protein that can be recognized by a commercially available antibody. A fusion protein may also be engineered to contain a cleavage site located between the NPG encoding sequence and the heterologous protein sequence, so that NPG may be cleaved and purified away from the heterologous moiety.

The protein may be produced using chemical methods to synthesize the amino acid sequence of NPG, or a fragment thereof. For example, peptide synthesis can be performed using various solid-phase techniques. (See, e.g., Roberge, J. Y. et al. (1995) Science 269:202-204.) Automated synthesis may be achieved, for example, using the ABI 431A peptide synthesizer (Perkin Elmer).

The newly synthesized peptide may be substantially purified by preparative high performance liquid chromatography. (See, e.g., Creighton, T. (1983) Proteins, Structures and Molecular Principles, W H Freeman and Co., New York, N.Y.) The composition of the synthetic peptides may be confirmed by amino acid analysis or sequencing (e.g., the Edman degradation procedure; Creighton, supra.) Additionally, the amino acid sequence of NPG, or any part thereof, may be altered during direct synthesis and/or combined using chemical methods with sequences from other proteins, or any part thereof, to produce a variant polypeptide.

In order to express a biologically active NPG, the nucleotide sequences encoding NPG or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding NPG and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview, N.Y.; and Ausubel, F. M. et al. (1989) Current Protocols in Molecular Biology, John Wiley & Sons, New York, N.Y.)

A variety of expression vector/host systems may be utilized to contain and express sequences encoding NPG. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus) or with bacterial expression vectors (e.g., Ti or pBR322 plasmids); or animal cell systems. The invention is not limited by the host cell employed.

The “control elements” or “regulatory sequences” are those regions of the vector, e.g., enhancers, promoters, 5′ and 3′ untranslated regions, translated regions, which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or PSPORT1 plasmid (Gibco BRL) and the like may be used. The baculovirus polyhedrin promoter may be used in insect cells. Promoters or enhancers derived from the genomes of plant cells (e.g., heat shock, RUBISCO; and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding NPG, vectors based on SV40 or EBV may be used with an appropriate selectable marker.

In bacterial systems, a number of expression vectors may be selected depending upon the use intended for NPG. For example, when large quantities of NPG are needed for the induction of antibodies, vectors which direct high level expression of fusion proteins that are readily purified may be used. Such vectors include, but are not limited to, the multifunctional E. coli cloning and expression vectors such as the BLUESCRIPT phagemid (Stratagene), in which the sequence encoding NPG may be ligated into the vector in frame with sequences for the amino-terminal Met and the subsequent 7 residues of β-galactosidase so that a hybrid protein is produced; pIN vectors (Van Heeke, G. and S. M. Schuster (1989) J. Biol. Chem. 264:5503-5509); and the like. PGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. (See, e.g., Ausubel et al., supra; and Grant et al. (1987) Methods Enzymol. 153:516-544.)

An insect system may also be used to express NPG. For example, in one such system, Autographa californica nuclear polyhedrosis virus (AcNPV) is used as a vector to express foreign genes in Spodoptera frugiperda cells or in Trichoplusia larvae. The sequences encoding NPG may be cloned into a non-essential region of the virus, such as the polyhedrin gene, and placed under control of the polyhedrin promoter. Successful insertion of NPG will render the polyhedrin gene inactive and produce recombinant virus lacking coat protein. The recombinant viruses may then be used to infect, for example, S. frugiperda cells or Trichoplusia larvae in which NPG may be expressed (Engelhard, E. K. et al. (1994) Proc. Nat. Acad. Sci. 91:3224-3227).

In mammalian host cells, a number of viral-based expression systems may be utilized. In cases where an adenovirus is used as an expression vector, sequences encoding NPG may be ligated into an adenovirus transcription/translation complex consisting of the late promoter and tripartite leader sequence. Insertion in a non-essential E1 or E3 region of the viral genome may be used to obtain a viable virus which is capable of expressing NPG in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Human artificial chromosomes (HACs) may also be employed to deliver larger fragments of DNA than can be contained and expressed in a plasmid. HACs of 6 to 10M are constructed and delivered via conventional delivery methods (liposomes, polycationic amino polymers, or vesicles) for therapeutic purposes.

Specific initiation signals may also be used to achieve more efficient translation of sequences encoding NPG. Such signals include the ATG initiation codon and adjacent sequences. In cases where sequences encoding NPG, its initiation codon, and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells which have specific cellular machinery and characteristic mechanisms for post-translational activities (e.g., CHO, HeLa, MDCK, HEK293, and W138), are available from the American Type Culture Collection (ATCC; Bethesda, Md.) and may be chosen to ensure the correct modification and processing of the foreign protein.

For long-term, high-yield production of recombinant proteins, stable expression is preferred. For example, cell lines stably expressing NPG can be transformed using expression vectors containing viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1980) Cell 22:817-23) genes which can be employed in tk⁻ or aprt⁻ cells, respectively. Also, antimetabolite, antibiotic, or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14), and genes which confer resistance to hygromycin and puromycin. Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). Recently, the use of visible markers has gained popularity with such markers as anthocyanins, and its substrate GUS, and luciferase and its substrate luciferin, being widely used not only to identify transformants, but also to quantify the amount of transient or stable protein expression attributable to a specific vector system (Rhodes, Calif. et al. (1995) Methods Mol. Biol. 55:121-131).

Antibodies

Antibodies to NPG may be generated using methods that are well known in the art. Such antibodies may include, but are not limited to, polyclonal, monoclonal, chimeric, single chain, Fab fragments, and fragments produced by a Fab expression library. Neutralizing antibodies, (i.e., those which inhibit dimer formation) are especially preferred for therapeutic use.

For the production of antibodies, various hosts including goats, rabbits, rats, mice, humans, and others, may be immunized by injection with NPG or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to NPG have an amino acid sequence consisting of at least five amino acids and more preferably at least 10 amino acids, and most preferably at least 15 amino acids. It is also preferable that they are identical to a portion of the amino acid sequence of the natural protein, and they may contain the entire amino acid sequence of a small, naturally occurring molecule. Short stretches of NPG amino acids may be fused with those of another protein such as keyhole limpet hemocyanin and antibody produced against the chimeric molecule.

Monoclonal antibodies to NPG may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce NPG-specific single chain antibodies. Antibodies with related specificity, but of distinct idiotypic composition, may be generated by chain shuffling from random combinatorial immunoglobulin libraries (Burton D. R. (1991) Proc. Natl. Acad. Sci. 88:11120-3).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. 86: 3833-3837; Winter, G. et al. (1991) Nature 349:293-299).

Antibody fragments which contain specific binding sites for NPG may also be generated. For example, such fragments include, but are not limited to, the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between NPG and its specific antibody. A two-site, monoclonal-based immunoassay utilizing monoclonal antibodies reactive to two non-interfering NPG epitopes is preferred, but a competitive binding assay may also be employed (Maddox, supra).

Uses

The present invention provides various methods for determining whether a compound can modulate the activity of NPG. The compound can be a substantially pure compound of synthetic origin combined in an aqueous medium, or the compound can be a naturally occurring material such that the assay medium is an extract of biological origin, such as, for example, a plant, animal, or microbial cell extract. The compound can also be a member of a compound library in which all members of the compound library are screened using the methods described below. The methods essentially entail contacting NPG or fragments thereof, with the compound under suitable conditions and subsequently determining if the compound modulates the activity of NPG. The compounds of interest can function as agonists or antagonists of NPG activity. NPG or fragments thereof, can be expressed on a cell or tissue, naturally or recombinantly, or immobilized by attachment to a solid substrate, e.g., nitrocellulose or nylon membrane, glass, beads, etc. Several high-throughput screening assays are known in the art. (See, e.g., Sittampalam, et al. (1997) Curr. Opin. Chem. Biol. 391; and Silverman, et al. (1998) Curr. Opin. Chem. Biol. 2:397-403.)

Transcription based assays that identify signals that modulate the activity of cell surface proteins, e.g., receptors, ion channels, etc., may be used to screen candidate compounds for their ability to stimulate reporter gene product expression and their potential to stimulate the expression of NPG.

One method tar identifying compounds that stimulate NPG promoter-controlled reporter gene expression comprises introducing into a cell a DNA construct that comprises NPG promoter operably linked to a reporter gene, mixing a test compound with the cell and measuring the level of expression of reporter gene product. A change in the level of expression of the reporter gene product indicates that the compound is capable of modulating the level of NPG expression. The reporter gene construct is preferably stably integrated into the chromosomal DNA of the cell, but is also functional for the purposes disclosed herein in the form of an extra-chromosomal element. The cell may be a eukaryotic cell, or any cell that contains the elements needed to express a structural gene under the regulatory influence of a mammalian gene promoter.

Other transcription based assays are well known in the art. (See, e.g., Zlokamik, et al. (1998) Science 279:84-88; Siverman, supra; and Gonzalez and Negulescu, (1998) Curr. Opin. Biotechnol. 9:624-631.) These transcription based assays asses the intracellular transduction of an extracellular signal using recombinant cells that are modified by introduction of a reporter gene under the control of a regulatable promoter.

A two-hybrid system-based approach can also be employed for compound screening, small molecule identification, and drug discovery. The underlying premise of the two-hybrid system, originally described in yeast by Fields and Song (1989) Nature 340:245-246, provides a connection between a productive protein-protein or protein-compound interaction pair of interest and a measurable phenotypic change in yeast. A reporter cassette containing an up-stream activation sequence which is recognized by a DNA binding domain, is operationally linked to a reporter gene, which when expressed under the correct conditions will generate a phenotypic change. The original two-hybrid system has recently been modified for applicability in high-throughput compound screening. (See, e.g., Ho et al. (1996) Nature 382:822-826; Licitra and Liu (1996) Proc. Natl. Acad. Sci. USA 93:12817-12821; and Young et al. (1998) Nature Biotech. 16:946-950.)

Assays for identifying compounds that modulate ion channel activity are practiced by measuring the ion channel activity when a cell expressing the ion channel of interest, or fragments thereof, is exposed to a solution containing the test compound and a ion channel selective ion and comparing the measured ion channel activity to the native ion channel activity of the same cell or a substantially identical control cell in a solution not containing the test compound. Methods for practicing such assays are known to those of skill in the art. (See, e.g., Mishina et al. (1985) Nature 313:364-369; and Noda, et al.

Ion channel activity can be measured by methods such as electrophysiology (two electrode voltage clamp or single electrode whole cell patch clamp), guanidinium ion flux assays, toxin-binding assays, and Fluorometric Imaging Plate Reader (FLIPR) assays. (See, e.g., Sullivan, et al. (1999) Methods Mol. Biol. 114:125-133; Siegel and Isacoff (1997) Neuron 19:1-20; and Lopatin, et al. (1998) Trends Pharmacol. Sci. 19:395-398.) An “inhibitor” is defined generally as a compound, at a given concentration, that results in greater than 50% decrease in ion channel activity, preferably greater than 70% decrease in ion channel activity, more preferably greater than 90% decrease in ion channel activity.

The binding or interaction of the compound with a receptor or fragments thereof, can be measured directly by using radioactively labeled compound of interest (see, e.g., Wainscott et al. (1993) Mol. Pharmacol. 43:419-426; and Loric, et al. (1992) FEBS Lett. 312:203-207) or by the second messenger effect resulting from the interaction or binding of the candidate compound. (See, e.g., Lazereno and Birdsall (1993) Br. J. Pharmacol. 109:1120-1127.) Modulation in receptor signaling can be measured using a detectable assay, e.g., the FLIPR assay. (See, e.g., Coward, P. (1999) Anal. Biochem. 270:242-248; Sittampalam, supra; and Gonzalez and Negulescu, supra.) Activation of certain receptors, in particular, GPCRs, can be measured an ³⁵S GTPγS binding assay. (See, e.g., Lazareno (1999) Methods Mol. Biol. 106:231-245.)

Alternatively, the candidate compounds can be subjected to competition screening assays, in which a known ligand, preferably labeled with an analytically detectable reagent, most preferably radioactivity, is introduced with the drug to be tested and the capacity of the compound to inhibit or enhance the binding of the labeled ligand is measured. Compounds are screened for their increased affinity and selectivity for the specific receptor or fragments thereof.

Candidate compounds are useful in the treatment or prophylaxis of disease states associated with the nervous system, in particular, neuropathic pain, perphipheral neuropathies, post-traumatic pain, post-surgical pain, pain associated with cancer, pain associated with chemotheraphy, and neurological disorders including, but not limited to cognitive disorders, such as Alzheimer's disease and dementia.

The polynucleotides of the present invention can be used to design antisense oligonucleotides that inhibit translation of mRNA encoding the NPG of the present invention. Synthetic oligonucleotides, or other antisense chemical structures are designed to bind to mRNA encoding NPG and inhibit translation of mRNA and are useful to inhibit expression of NPG. This invention provides a means to alter levels of expression of NPG by the use of a synthetic antisense oligonucleotide (SAO) which inhibits translation of mRNA encoding these receptors.

The SAO is designed to be capable of passing through cell membranes in order to enter the cytoplasm of the cell by virtue of physical and chemical properties of the SAO which render it capable of passing through cell membranes (e.g. by designing small, hydrophobic SAO chemical structures) or by virtue of specific transport systems in the cell which recognize and transport the SAO into the cell. In addition, the SAO can be designed for administration only to certain selected cell populations by targeting the SAO to be recognized by specific cellular uptake mechanisms which binds and takes up the SAO only within certain selected cell populations. For example, the SAO may be designed to bind to NPG which are found only in certain cell types.

The SAO is also designed to recognize and selectively bind to the target mRNA sequence, which may correspond to a sequence contained within the sequences of SEQ ID NO:1, 3, 5, 7, 9, 11, 13, or 15 by virtue of complementary base pairing to the mRNA. Finally, the SAO is designed to inactivate the target mRNA sequence by any of three mechanisms: 1) binding to the target mRNA and thus inducing degradation of the mRNA by intrinsic cellular mechanisms such as RNAse digestion; 2) inhibiting translation of the mRNA target by interfering with the binding of translation-regulating factors or of ribosomes; or 3) inclusion of other chemical structures, such as ribozyme sequences or reactive chemical groups, which either degrade or chemically modify the target mRNA.

Synthetic antisense oligonucleotide drugs have been shown to be capable of the properties described above when directed against mRNA targets. (See, e.g., Cohen (1989) Trends in Pharm. Sci. 10:435; and Weintraub (1990) Sci. Am. 262:40-46.) In addition, coupling of ribozymes to antisense oligonucleotides is a promising strategy for inactivating target mRNA. (See, e.g., Sarver et al. (1990) Science 247:1222.)

Diagnostics and Kits

The present invention contemplates use NPG polynucleotides, polypeptides, and antibodies in a variety of diagnostic methods kits. Typically the kit will have a compartment containing either a defined NPG polypeptide, polynucleotide, or a reagent which recognizes one or the other, e.g., antigen fragments or antibodies. Additionally the kit will include the reagents needed to carry out the assay in a separate compartment as well as instructions for use and proper disposal.

A variety of protocols including ELISA, RIA, and FACS for measuring NPG are known in the art and provide a basis for diagnosing altered or abnormal levels of NPG expression. Normal or standard values for NPG expression are established by combining body fluids or cell extracts taken from normal mammalian subjects, preferably human, with antibody to NPG under conditions suitable for complex formation. The amount of standard complex formation may be quantified by various methods, but preferably by photometric, means. Quantities of NPG expressed in control and disease samples from biopsied tissues are compared with the standard values. Deviation between standard and subject values establishes the parameters for diagnosing disease.

In another embodiment of the invention, the polynucleotides encoding NPG may be used for diagnostic purposes. The polynucleotides which may be used include, oligonucleotide sequences, complementary RNA and DNA molecules, and PNAs. The polynucleotides may be used to detect and quantitate gene expression in biopsied tissues in which expression of NPG may be correlated with disease. The diagnostic assay may be used to distinguish between absence, presence, and excess expression of NPG, and to monitor regulation of NPG levels during therapeutic intervention.

In one aspect, hybridization with PCR probes which are capable of detecting polynucleotide sequences, including genomic sequences, encoding NPG or closely related molecules, may be used to identify nucleic acid sequences which encode NPG. The specificity of the probe, whether it is made from a highly specific region, e.g., 10 unique nucleotides in the 5′ regulatory region, or a less specific region, e.g., especially in the 3′ coding region, and the stringency of the hybridization or amplification (maximal, high, intermediate, or low) will determine whether the probe identifies only naturally occurring sequences encoding NPG, alleles, or related sequences.

Probes may also be used for the detection of related sequences, and should preferably contain at least 50% of the nucleotides from any of the NPG encoding sequences. The hybridization probes of the subject invention may be DNA or RNA and derived from the nucleotide sequence of SEQ ID NO:1, 3, 5, 7, 9, 11, 13, or 15, or from genomic sequence including promoter, enhancer elements, and introns of the naturally occurring NPG.

Means for producing specific hybridization probes for DNAs encoding NPG include the cloning of nucleic acid sequences encoding NPG or NPG derivatives into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as ³²P or ³⁵S, or enzymatic labels, such as alkaline phosphatase coupled to the probe via avidin/biotin coupling systems, and the like.

Polynucleotide sequences encoding NPG may be used for the diagnosis of disease states associated with the nervous system, in particular, neuropathic pain, perphipheral neuropathies, post-traumatic pain, post-surgical pain, pain associated with cancer, pain associated with chemotheraphy; and neurological disease states including, but not limited to cognitive disease states, such as Alzheimer's disease and dementia.

In order to provide a basis for the diagnosis of disease states associated with expression of NPG, a normal or standard profile for expression is established. This may be accomplished by combining body fluids or cell extracts taken from normal subjects, either animal or human, with a polynucleotide sequence, or a fragment thereof, which encodes NPG, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from subjects who are symptomatic for disease. Deviation between standard and subject values is used to establish the presence of disease.

Once a disease is established and a treatment protocol is initiated, hybridization assays may be repeated on a regular basis to evaluate whether the level of expression in the subject begins to approximate that which is observed in the normal subject. The results obtained from successive assays may be used to show the efficacy of treatment over a period ranging from several days to months.

Additional diagnostic uses for oligonucleotides designed from the sequences encoding NPG may involve the use of PCR. Such oligomers may be chemically synthesized, generated enzymatically, or produced in vitro. Oligomers will preferably consist of two nucleotide sequences, one with sense orientation (5′ to 3′) and another with antisense (3′ to 5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Methods which may also be used to quantitate the expression of NPG include radiolabeling or biotinylating nucleotides, coamplification of a control nucleic acid, and standard curves onto which the experimental results are interpolated. (See, e.g, Melby, P. C. et al. (1993) J. Immunol. Methods, 159:235-244; and Duplaa, C. et al. (1993) Anal. Biochem. 212:229-236.) The speed of quantitation of multiple samples may be accelerated by running the assay in an ELISA format where the oligomer of interest is presented in various dilutions and a spectrophotometric or calorimetric response gives rapid quantitation.

In another embodiment of the invention, the nucleic acid sequences which encode NPG may also be used to generate hybridization probes which are useful for mapping the naturally occurring genomic sequence. The sequences may be mapped to a particular chromosome, to a specific region of a chromosome or to artificial chromosome constructions, such as human artificial chromosomes (HACs), yeast artificial chromosomes (YACs), bacterial artificial chromosomes (BACs), bacterial P1 constructions, or single chromosome cDNA libraries. (See, e.g., Price, C. M. (1993) Blood Rev. 7:127-134, and Trask, B. J. (1991) Trends Genet. 7:149-154.)

Fluorescent in situ hybridization (FISH) may be correlated with other physical chromosome mapping techniques and genetic map data. (See, e.g., Verma et al. (1988) Human Chromosomes: A Manual of Basic Techniques, Pergamon Press, New York, N.Y.) Examples of genetic map data can be found in various scientific journals or at Online Mendelian Inheritance in Man (OMIM). Correlation between the location of the gene encoding NPG on a physical chromosomal map and a specific disease, or predisposition to a specific disease, may help delimit the region of DNA associated with that genetic disease. The nucleotide sequences of the subject invention may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region (see, e.g., Gatti, R. A. et al. (1988) Nature 336:577-580), any sequences mapping to that area may represent associated or regulatory genes for further investigation. The nucleotide sequence of the subject invention may also be used to detect differences in the chromosomal location due to translocation, inversion, etc. among normal, carrier, or affected individuals.

All patents, patent applications, and publications mentioned herein, whether supra or infra, are each incorporated by reference in its entirety. The broad scope of this invention is best understood with reference to the following examples, which are not intended to limit the invention to the specific embodiments described below.

### EXAMPLES

Some of the standard methods are described or referenced, e.g., in Maniatis et al. (1982) Molecular Cloning, A Laboratory Manual, Cold Spring Harbor Laboratory, Cold Spring Harbor Press; Sambrook et al. (1989) Molecular Cloning: A Laboratory Manual, (2d ed.), vols. 1-3, CSH Press, N.Y.; or Ausubel et al. (1987 and Supplements) Current Protocols in Molecular Biology, Greene/Wiley, New York; Innis et al. (eds.)(1990) PCR Protocols: A Guide to Methods and Applications Academic Press, N.Y. Methods for protein purification include such methods as ammonium sulfate precipitation, column chromatography, electrophoresis, centrifugation, crystallization, and others. See, e.g., Ausubel et al. (1987 and periodic supplements); Deutscher (1990) “Guide to Protein Purification” in Methods in Enzymology, vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering Principle and Methods 12:87-98, Plenum Press, N.Y.; and Crowe et al. (1992) OIAexpress: The High Level Expression & Protein Purification System QUIAGEN, Inc., Chatsworth, Calif.

### Example I

### Double-Stranded cDNA Synthesis from Rat Dorsal Root Ganglia (DRG) Poly(A)⁺ RNA

Dorsal root ganglia from L4-L5 spinal levels of 10 Sprague-Dawley rats were removed from the animals and frozen in liquid nitrogen. Poly(A)⁺ RNAs were extracted using MICRO-FASTTRACK RNA isolation kit (Invitrogen, San Diego, Calif., USA) and stored at −80° C. Reverse transcription was carried out with 100 ng of DRG poly(A)+RNA and the SMART PCR cDNA synthesis kit (CLONTECH Laboratories Inc., Palo Alto, Calif., USA). The resulting double-stranded cDNA was used as tester cDNA in the subsequent subtraction. The driver cDNAs were prepared from poly (A⁺) RNA isolated from liver, skeletal muscle, heart, spleen, and kidney (CLONTECH) as described above.

### Example II

### Isolation of cDNA Clones that are Differentially Expressed in Dorsal Root Ganglia (DRG).

A subtracted cDNA library was constructed for dorsal root ganglia isolated 10 days after peripheral nerve injury (Diatchenko et al. (1996) Proc. Natl. Acad. Sci. 93:6025-6030; and Jin et al. (1997) BioTechniques 23:10841086) using the PCR-SELECT cDNA subtraction kit (CLONTECH). The driver cDNAs were made using mRNAs isolated from skeletal muscle, kidney, liver, spleen and heart. Also included was DRG mRNA from Day 0 (i.e. prior to nerve injury) in the preparation of driver cDNAs to enrich genes that are up-regulated in response to the peripheral axotomy.

### Example III

### RNA/DNA Dot Blot Analysis

Dot blots were prepared using poly(A)⁺ RNAs from skeletal muscle, kidney, liver, spleen, heart, and brain, and SMART PCR-generated cDNA (Clontech) from DRG poly(A)⁺ RNA. After normalization of each dot to both glyceraldehyde-3phosphate dehydrogenase (G3PDH) and β-actin, 0.1-0.25 ug of each poly(A)⁺RNA or 0.05 ug of SMART PCR-generated DRG cDNA was mixed with 3% Ficoll and 0.1% bromophenol blue (20 mM NaOH was used for DNA samples or without 20 mM NaOH for RNA samples). Each mixture was then applied to a nylon membrane at 1 ul per dot using a HYDRA-96 automatic dispenser (Robbins Scientific, Sunnyvale, Calif., USA). The membranes were UV cross-linked (Stratagene, La Jolla, Calif., USA) and neutralized in a solution containing 0.1 M Tris (pH 7.4) and 2× saline sodium citrate (SSC) before hybridization. Inserts from selected cDNA clones were amplified by PCR, labeled with ³²P-dCTP (Sambrook, supra) and used as probes in hybridization. Hybridization was carried out using EXPRESSHYB hybridization solution (Clontech) at 65° C. overnight. The next morning the filters were washed twice in 2× SSC and 0.1% SDS at 65° C. for 15 min., twice with 0.1× SSC and 0.1% SDS at 65° C. for 15 min. and were subsequently exposed to BIOMAX MS autoradiography film (Kodak) for 1-3 days. Eight out of 12 cDNA clones randomly selected from the Day 10 subtracted DRG cDNA library were either DRG-specific or DRG- and/or brain-enriched, indicating a subtraction efficiency of about 67%. Subsequently, 410 random cDNA clones were picked and sequenced using an ABI 373 automatic sequencer (Applied Biosystems, Inc., Foster City, Calif.). The resulting nucleotide sequences were compared with those in the databases of GenBank (Gaithersburg, Md.), EMBL (Heidleberg, Germany) and LIFESEQ (Incyte Pharmaceuticals, inc., Palo Alto, Calif.). Approximately 63.7% of these sequences corresponded to known genes and 37.3% were unknown. To further analyze specific neuropathic pain-related genes, 36 cDNA clones were chosen for differential expression analysis on an array.

### Example IV

### cDNA Array Preparation

Inserts of selected cDNA clones were amplified using nested primers (Jin, supra) with the PCR protocol. 100 ul of each PCR product was precipitated with ethanol and resuspended at a final concentration of 100 ng/ul in H20. To prepare for arraying, each sample was mixed with an equal volume of 0.6 N NaOH/15% Ficoll/0.5% bromophenol blue. One ul of each denatured DNA sample was transferred onto a nylon membrane using a HYDRA-96 automatic dispenser (Robbins Scientific). The nylon membrane was UV cross-linked (Stratagene) and neutralized in 0.5 M Tris-HCl (pH 7.5).

Radioactively labeled cDNA probes were prepared as described in Sambrook, supra, with some modifications. In the first strand cDNA synthesis step, a 25 ul reaction contained 0.1-0.2 ug of poly(A)⁺ mRNA, oligo (dT) (25-mer), and random primer (6-mer), IX first-strand cDNA synthesis buffer, 2 mM DTT, 200 uM dATP, 200 uM dGTP, 200 uM dTTP, 0.2 uM dCTP, 100 μCi ³²P-dCTP, and 100 units of reverse transcriptase (GIBCO, Gaithersburg, Md.). The reverse transcription was carried out at 42° C. for 1 hour, extracted with phenol:chloroform, precipitated with ethanol, and resuspended in 15 ul of TE. Samples were mixed with 1.5 ul of 2.5 N NaOH, and incubated for 30 minutes at 68° C. to hydrolyze the RNA. After cooling to room temperature, the samples were neutralized by adding of 2 ul of 2M Tris (pH 7.5) and 1.5 ul of 2.5 N HCl. The resulting cDNAs were purified by passing through CHROMA SPIN-200 columns (CLONTECH), co-precipitated with glycogen in ethanol, and resuspended in 100 ul of H₂0.

³²P-labeled cDNA probes were heat-denatured and hybridized to the cDNA arrays in EXPRESSHYB hybridization solution (CLONTECH). The nylon membranes were washed as described above and then scanned by a PHOSPHORIMAGER (Molecular Dynamics, Sunnyvale, Calif., USA). The resulting images were then processed and analyzed using ATLAS VISION software (CLONTECH), which produces a pseudocolor representation of relative gene expression levels. On a single array, the hybridization intensity of each cDNA clone was normalized to that of the internal control clone G5, and the relative expression level of each cDNA clone at different time points was represented in a pseudocolor scale corresponding to the ratio of a normalized readout relative to that at Day 0 (i.e., the readout at Day 0 is arbitrarily set at 1).

Hybridization signals were observed for 85% of the rat cDNA array elements, but not to the negative control, a plasmid DNA, (data not shown). All highly and moderately moderately abundant transcripts, as well as a few rare transcripts were detected. At least 12 cDNA clones that were apparently up-regulated in response to the chronic constriction injury were found. These gene expression levels remained at high levels between 7 to 14 days following the nerve injury, and then subsequently declined towards the normal state.

### Example V

### Sequence Extension of Differentially Regulated NPG cDNAs

Some NPG clones were extended to full length clones using a modified Rapid Amplification of cDNA Ends (RACE) procedure. (See, Ausubel, supra.) Two rounds of PCR were run using EXPAND long range PCR kit (Boeringer-Mannheim, Indianapolis, Ind.) Alternatively, standard library screening using the partial cDNAs described above, was performed. (See, Maniatis, supra; and Ausubel, supra.)

### Example VI

### Isolation of NPG-8

NPG-8 was isolated using a 5955 bp fragment of NPG-6 to search the LifeSeq database (Incyte Pharmaceuticals, Palo Alto, Calif.). Several clones, including clone id. 3600812) were assembled into a 1.5 kb contiguous sequence. PCR primers designed from this assembled sequence were then used to amplify a MARATHON cDNA library (Clontech) from human brain. Using the SMART RACE cDNA amplification kit (Clontech), larger extension products were isolated. One 3.1 kb product, MT-6, was used for subsequent extensions and sequencing, and has yielded a 19 kb sequence.

### Example VII

### In situ Hybridization of NPG-6

3′ untranslated sense and anti-sense PCR fragment (253 bp) was labeled with ³²P was labeled using the RIBOPROBE system (Promega) in vitro transcription reaction. (See, e.g., Lu and Gillet (1994) Cell Vision 1:169-176.) Fresh frozen tissues were cryostat cut at 12 μm and thaw mounted on aminosilane coated slides. Tissue sections were pretreated as follows (all steps at room temperature unless otherwise noted): 4% paraformaldehyde, 0.05% glutaraldehyde for 5 minutes at 4° C.; PBS for 2 minutes; acetic anhydride in 0.1 M TEA for 5 minutes; PBS for 2 minutes; 2× SSC for 2 minutes; and dehydration in ascending concentrations of ethanol.

Sections were prehybridized for 1 hour at 42° C. in a covered hybridization box lined with moist filter paper saturated with 4× SSC and 50% formamide. Probe was applied to slides and allowed to incubate overnight at 60° C. Slides were washed in 2× SSC for 5 minutes two times; subjected to RNase digestion (20 μg/ml RNase A in RNase buffer) for 30 mintues at 37° C.; rinsed two times in RNase buffer for minutes at 37° C.; four changes of 2× SSC for 30 minutes each; four changes of 1× SSC for 15 minutes each; 0.1× SSC fro 1 hour at 65° C.; and 0.1× SSC for 10 minutes twice. Washed slides were dehydrated in ascending concentration of ethanol in 0.3M NH₄OAC, dried and exposed to βMAX HYPERFILM (Amersham) for 10 days.

Although the foregoing invention has been described in some detail by way of illustration and example for purposes of clarity and understanding, it will be obvious that certain changes and modifications may be practiced within the scope of the appended claims.

What is claimed is:

1. An isolated polynucleotide encoding a NPG polypeptide.

2. The polynucleotide of claim 1, wherein the polynucleotide encodes a NPG polypeptide comprising the amino acid sequence of SEQ ID NO:2, 4, 6, 8,10,12, 14, or 16.

3. The polynucleotide of claim 2, wherein the polynucleotide is detectably labeled.

4. An isolated polynucleotide which is the complement of the polynucleotide of claim 2.

5. The isolated polynucleotide of claim 4, wherein the polynucleotide is detectably labeled.

6. The polynucleotide of claim 2, wherein the polynucleotide comprises the nucleic acid sequence of SEQ ID NO:1, 3, 5, 7, 9, 11, 13, or 15.

7. An expression vector comprising the polynucleotide of claim 2.

8. A host cell comprising the expression vector of claim 7.

9. The host cell of claim 8, wherein the host cell is a prokaryotic cell.

10. The host cell of claim 8, wherein the host cell is a eukaryotic cell.

11. A method of producing an NPG polypeptide comprising:
a) culturing the host cell of claim 8 under conditions suitable for expression of the NPG polypeptide; and
b) recovering the polypeptide from the host cell.

12. A method of detecting a polynucleotide encoding an NPG polypeptide in a sample containing nucleic acid material comprising:
a) contacting the sample with the polynucleotide of claim 4 under conditions suitable for formation of a hybridization complex; and
b) detecting the complex, wherein the presence of the complex is indicative of the presence of the polynucleotide encoding the polypeptide in the sample.

13. A diagnostic test kit comprising:
a) the polynucleotide of claim 6; and
b) instructions for conducting the diagnostic test.

14. A method of screening for a compound that modulates NPG activity, the method comprising:
a) contacting NPG, or fragment thereof with the compound;
b) detecting modulation of NPG activity.

15. The method of claim 14, wherein NPG is:
a) expressed on a cell or tissue; or
b) immobilized on a solid support.

16. The method of claim 14, wherein the compound is:
a) an antagonist of NPG activity;
b) an agonist of NPG activity.

17. An isolated NPG polypeptide comprising the amino acid sequence of SEQ ID NO:2, 4, 6, 8, 10, 12, 14, or 16.

18. The polypeptide of claim 17, wherein the polypeptide is:
a) recombinantly produced; or
b) synthetically produced.

19. An isolated antibody which specifically binds to the polypeptide of claim 17.

20. A transgenic nonhuman mammal comprising the polynucleotide of claim 2.

21. A transgenic nonhuman mammal comprising the polynucleotide of claim 4 capable of hybridizing to a polynucleotide encoding NPG, thereby reducing expression of NPG.
